(19) United States
(12) Patent Application Publication
(10) Pub. No.: US 2006/0018880 A1
(43) Pub. Date: Jan. 26, 2006

(54) Methods and compositions for delivery of an enhanced response to antigenic substrates

(75) Inventors: Timothy L. Ratliff, Iowa City, IA (US); David W. Lubaroff, Iowa City, IA (US); D. Robert Siemens, Kingston (CA); Bennett D. Elzey, St. Louis, MO (US)

Correspondence Address: MCKEE, VOORHEES & SEASE, P.L.C., 801 GRAND AVENUE, SUITE 3200, DES MOINES, IA, 50309-2721, US

(73) Assignee: University of Iowa Research Foundation, Oakdale, IA (US)

(21) Appl. No.: 11/041,851

(22) Filed: Jan. 24, 2005

Related U.S. Application Data
(63) Continuation-in-part of application No. 09/511,052, filed on Feb. 23, 2000, now abandoned.
(60) Provisional application No. 60/121,644, filed on Feb. 24, 1999.

(51) Int. Cl. A61K 48/00; C12N 15/86

(52) U.S. Cl. 424/93.2; 514/44; 435/456

(57) ABSTRACT

A novel immunostimulating delivery system is disclosed which provides for generation of a specific antibody response and specific T cell response to delivered antigens. According to the invention, the antigen or a nucleotide encoding the same is contained within, impregnated in or associated with a biocompatible collagen based carrier composition. Methods and compositions for practicing the same are disclosed.

### CROSS-REFERENCE TO RELATED APPLICATION

This application claims the benefit of U.S. application Ser. No. 09/511,052 filed Feb. 23, 2000 which claims the benefit of U.S. Provisional Application Ser. No. 60/121,644 filed Feb. 24, 1999, the disclosures of which are hereby incorporated by reference.

### GRANT REFERENCE

The work presented in this application was supported in part by grants from the Carver Trust Foundation and the American Cancer Society (#TRPN-98-335-01-CIM), the government may gave certain rights in this invention.

### BACKGROUND OF THE INVENTION

Vaccines comprise antigens or combinations of antigens which, when administered to a warm-blooded animal, prevent, ameliorate, or treat disease. Originally, vaccines for infectious diseases comprised whole, attenuated or killed microbes. It was soon discovered, however, that only a few proteins or protein fragments of a microbe or cell stimulated a protective immune response, and, in fact, inclusion of extraneous materials from the whole cell hindered the immune response. Thus, vaccine development focused on identifying the particular protein, protein fragment, epitope and DNA segment encoding that epitope which elicited the protective immune response. As antigen identification became more precise however, vaccine efficiency declined. Identified antigens were often small molecules unable to be recognized by antigen processing cells. It was therefore necessary to combine these antigens with substances which enhance the antigenicity of the antigen and give a superior immune response. These substances are adjuvants.

Adjuvants work by several means. Some assist in the presentation of antigen to antigen processing cells (APC). Oil-in-water emulsions, water-in-oil emulsions, liposomes and microbeads each assist in presenting antigen to APC. Small antigens or haptens are often linked to larger, immunogenic proteins or polysaccharides to facilitate recognition by the APC. Certain adjuvants have a depot effect holding antigen in place until the body has an opportunity to mount an immune response. Other adjuvants stimulate the immune system generally augmenting the specific response mounted to the antigen.

The most widely used antigen, the attenuated lipid A derivatives (ALD) monophosphoryl lipid A (MLA) and 3-deacylated monophosphoryl lipid A (3D-MLA) are potent immunological adjuvants used in prophylactic vaccines for infectious disease and therapeutic vaccines for the treatment of cancerous tumors and chronic infections. MLA and 3D-MLA are modified forms of the bacterial endotoxin lipopolysaccharide (LPS) and are known and described in U.S. Pat. Nos. 4,436,727 and 4,912,094, respectively. MLA and 3D-MLA induce both a humoral antibody response and a cell-mediated immune response in patients administered the compounds with an antigen.

Several polynucleotides have been extensively evaluated as immunostimulating compounds for adjuvants. Perhaps the best example is poly (I,C) which is a potent inducer of IFN production as well as a macrophage activator and inducer of NK activity (Talmadge, J. E., J. Adams, H. Phillips, M. Collins, B. Lenz, M. Schneider, E. Schlick, R. Ruffmann, R. H. Wiltrout, and M. A. Chirigos, 1985). These types of adjuvants are particularly desirable as the field of gene therapy has allowed for not only presentation of the antigen protein to a cell, but instead for presentation of a nucleotide vector which encodes the antigen protein to a cell. The antigen is then “manufactured” by the transfected cell, theoretically evading the immediate elimination of the foreign antigen before a specific immune response is generated. Unfortunately, toxic side effects have thus far prevented poly (I,C) from becoming a useful therapeutic agent.

The present invention involves a carrier composition for delivery of primarily nucleotide, although not limited thereto, encoding antigen vaccines, which increases the immune response observed. One particular embodiment involves the use of the carrier and nucleotide composition as a cancer vaccine.

Numerous substances have been employed as a carrier composition to enhance and sustain the delivery of soluble products to both neoplastic and non-neoplastic tissue. A polymer-based paste has been found to enhance local delivery of chemotherapeutic agents and decrease recurrence rates at tumor resection sites (Hunter, W. L., Burt, H. M., and Machine, L. Local delivery of chemotherapy—a supplement to existing cancer treatments—a case for surgical pastes and coated stents. Adv. Drug Delivery Rev. 26 (2-3):199-207, 1997). A fibrin- and gelatin-based drug delivery system has been shown to more slowly release and improves the therapeutic effects of antibiotics (Park, M S, Kim, YB Sustained release of antibiotic from a fibrin-gelatin-antibiotic mixture. Laryngoscope, 107 (10):1378, 1997). Poloxamer 407 has been shown to improve the delivery of adenoviral vectors in vascular smooth muscle based on β-galactosidase reporter gene expression (Feldman, L. J., Pastore, C. J, Aubailly, N., Kearney, M., Chen, D., Perricaudet, M., Steg, P. G., and Isner, J. M. Improved efficiency of arterial gene transfer by use of poloxamer 407 as a vehicle for adenoviral vectors. Gene Ther. 4:189-198, 1997). Gelfoam®, an absorbable gelatin sponge primarily used as an intraoperative hemostatic agent, has also been used to deliver a number of different compounds, including insulin (Park, M. S. et al.) as well as various cytokines (Lee, Y. C., Simamora, P. and Yalkowsky, S. H. Systemic delivery of insulin via an enhancer-free ocular device. J. of Pharm. Sci. 86 (12):1361-1364, 1997) and growth factors (Segal, D. H., Germano, I. M. and Berderson, J. B. Effects of basic fibroblast growth factor on in vivo cerebral tumorigenesis in rats. Neurosurgery, 40 (5):1027, 1997), in order to improve and sustain delivery.

These types of agents have been employed with nucleotide expression constructs on a limited basis. To date all research has been limited to repair or regeneration protocols where the matrix carrier is used to facilitate infiltration of fibroblasts or as scaffolding to promote wound healing. For example, U.S. Pat. No. 5,763,416 to Jeffrey Bonadio and Steven Goldstein, “Gene Transfer into Bone Cells and Tissues” discloses the use of gene delivery matrices for bone repair and regeneration in vivo. The matrix functions as a scaffold or a “biofiller” that provides a structure for developing bone and cartilage. The application also notes that any matrix formed from a variety of material presently used for implanted medical applications can be used. See also International Patent Application No. WO97/38729 and PNAS 93:5753-5758, 1996, “Stimulation of new bone formation by direct transfer of osteogenic plasmid genes”. Despite these repair and regeneration protocols involving use of a collagen matrix to form structural features, the concept of use of a collagen based carrier to act as an adjuvent increasing the immune response observed to nucleotides has never been postulated.

For the foregoing reasons, a need exists for a way to efficiently deliver, enhance the immune response to, and enhance expression of genes of nucleotide expression vectors for immunogenic (vaccine) protocols. Applicants have identified unique properties present in collagen or its derivatives that provide for increased immune response in vectors which are delivered by the methods and compositions of the invention, thus meeting this need in the art.

Another object of the invention is to provide a composition which enhances expression of the gene product of vectors, particularly viral vectors.

A further object of the invention is to provide a composition which enhances immune response observed to an introduced nucleotide or protein vaccine.

Yet another object of the invention is to provide a method for immunizing against and providing a protective response against tumors which comprises preimmunizing and animal according to the teachings herein.

Yet another object involves the pre-immunization and later treatment with cytokines which act synergistically to eradicate cancer cells upon introduction of the same.

These and other objects, features, and advantages will become apparent after review of the following description and claims of the invention which follow.

### SUMMARY OF THE INVENTION

The present invention seeks to utilize genetic engineering techniques to provide a recombinant nucleotide expression system delivery composition which unexpectedly provides for increased expression levels of the polynucleotide as well as the continued persistence of the expression system in vivo and thus improved efficiency. The system also provides for antigen-specific protective immunity for vaccine or other immunogenic protocols.

In one aspect, the invention provides a recombinant expression system contained within, impregnated in or associated with a biocompatible collagen based carrier composition.

As used herein the term “collagen carrier” shall mean any biocompatible composition which has the ability to be adsorbed, absorbed or otherwise maintain DNA and which has the ability to activate or interact with the clotting cascade in a similar manner to collagen. See, Alberio L., “Review Article “Platelet-collagen interactions:membrane receptors and intracellular signaling pathways”, Eur. J. Clin. Invest. 1999 Dec. 29 (12) 1066-1076. This includes collagen or any active derivative thereof or any other hemostatic factors which are collagen-like as further defined and described herein. This can include gelatin based agents which may or may not be cross-linked such as matrix or sponge formulations. See for example, PCT International Publication WO97/38729 the disclosure of which is incorporated by reference.

According to the invention, anti-tumor vector expression systems absorbed into a collagen carrier composition resulted in markedly enhanced delivery of the vector to pre-established subcutaneous tumor nodules. The collagen carrier material must have the ability to adsorbed, absorbed or otherwise maintain contact with a polynucleotide. Surprisingly, the inventors also discovered that gene expression of the collagen carrier/nucleotide system was quantitatively enhanced compared to expression of the system alone. Surprisingly, this carrier also resulted in increased expression when powdered collagen was used, a carrier which does not have the matrix features emphasized in prior regeneration protocols.

The term “expression system” is used herein to refer to a genetic sequence which includes a protein encoding region which is operably linked to all of the genetic signals necessary to achieve expression of the protein encoding region. Traditionally, the expression system will include a regulatory element such as a promoter or enhancer, to increase transcription and/or translation of the protein encoding region, or to provide control over expression. The regulatory element may be located upstream or downstream of the protein encoding region, or may be located at an intron (noncoding portion) interrupting the protein encoding region. Alternatively it is also possible for the sequence of the protein encoding region itself to comprise regulatory ability.

The term “functional equivalent” refers to any derivative which is functionally substantially similar to the referenced sequence or protein. In particular the term “functional equivalent” includes derivatives in which nucleotide base(s) and/or amino acid(s) have been added, deleted or replaced without a significantly adverse effect on biological function and which will hybridize under high conditions of stringency according to protocols known in the art and disclosed in Maniantis et. al., “Molecular Cloning” cold Spring Harbor Press, (1989).

As used herein the term “therapeutic gene” shall be interpreted in include any nucleotide sequence, the expression of which is desired in a host cell. This can include any genetic engineering protocol for introduction of such sequence which would benefit from increased expression, and improved retention time and includes antisense type strategies, diagnostic protocols, immune stimulating agents such as vaccines, or gene therapy.

Thus the invention in one embodiment includes a recombinant expression system which comprises a therapeutic nucleotide sequence, the expression of which is desired in a cell and a collagen carrier. The invention further encompasses vector delivery compositions containing the recombinant expression delivery system defined above, cells transformed with such vectors, and genetic engineering protocols using these components.

### DESCRIPTION OF THE FIGURES

FIG. 1A is a graph of the percentage (±standard deviation) of β-gal expressing RM-1 cells detected 48 hrs. after in vitro infection with ALVAC-lacZ.

FIG. 1B is a graph of the luciferase activity of MB-49 cell lysates 48 hrs. after in vitro infection with ALVAC-luciferase. No significant difference between gelatin sponge matrix delivery and direct infusion of fluid-phase product (p=0.46, Wilcoxon rank sum test).

FIG. 2A is a graph of the mean luciferase activity (±standard deviation) of harvested subcutaneous MB-49 tumors 48 hrs. after infection by ALVAC-luciferase delivered in vitro by different matrices. Significant differences seen between the gelatin sponge matrix delivery and fluid-phase injection (p=0.03, Wilcoxon rank sum test).

FIG. 2B is a graph of luciferase activity in RM-1 subcutaneous tumors injected with ALVAC-luciferase at different virion concentrations.

FIG. 3 is a graph of luciferase activity of heterotopic RM-1 tumor nodules after injection by ALVAC-luciferase. Tumors were harvested at various times after injection. Figures represent combined data from two separate experiments. Statistically significant (p<0.05, Wilcoxon rank sum test) difference was found between the gelatin matrix delivered groups and the fluid-phase delivered groups except at 96 hours (p=0.058).

FIG. 4A is a histopathology section of heterotopic RM-1 tumor nodules after infection with 3×10⁶ pfu ALVAC-GFP or ALVAC-lacZ showing limited and localized GFP expression demonstrated under fluorescent microscopy when ALVAC-GFP delivered by fluid-phase injection.

FIG. 4B is a histopathology section of heterotopic RM-1 tumor nodules after infection with 3×10⁶ pfu ALVAC-GFP or ALVAC-lacZ showing greater gene expression and wider distribution seen when ALVAC-GFP vector is delivered by the gelatin sponge matrix.

FIG. 4C is a histopathology section of heterotopic RM-1 tumor nodules after infection with 3×10⁶ pfu ALVAC-GFP or ALVAC-lacZ showing ALVAC-lacZ infected tumor stained with X-gal and counter stained with Nuclear Fast Red. Limited β-galactosidase expression seen in needle tract after injection of the fluid-phase product (original magnification×63).

FIG. 4D is a histopathology section of heterotopic RM-1 tumor nodules after infection with 3×10⁶ pfu ALVAC-GFP or ALVAC-lacZ showing lower power magnification of tumor infected with ALVAC-lacZ vector delivered by gelatin sponge matrix demonstrating qualitatively higher gene expression with greater distribution (original magnification×25).

FIG. 5 is a graph of the tumor inhibition effect of pre-established subcutaneous nodules infected by ALVAC virus encoding murine IL-2, IL-12, and TNF-α delivered either by the gelatin sponge matrix or by the fluid-phase product. Controls include matrix only, parental ALVAC virus, and no treatment groups. Statistically significant difference in tumor volume was found at days 2, 6, 8, 11 and 13 between matrix delivered virus and the control groups (p<0.001, one-way ANOVA).

FIG. 6A is a graph of the outgrowth of tumor nodules in individual mice with tumor inhibition and regression demonstrated when treatment virus was delivered by the gelatin sponge matrix.

FIG. 6B is a graph of the outgrowth of tumor nodules in individual mice with delivery of the virus in the fluid-phase.

FIG. 7 is a survival curve of C57BL-6 mice after tumor outgrowth study. Statistically significant survival advantage for those mice treated with intra-tumoral recombinant ALVAC when delivered by gelatin sponge matrix compared to fluid-phase injection and controls (p=0.015, Cox Proportional Hazards Regression Model).

FIG. 8. Transrectal ultrasound of the prostate during intraprostatic injection of carbon black. Sagittal view, 7.5 MHz. (A) Needle can be visualized along path of biopsy markers. (B) Hyperechoic lesion observed upon injection of biomarker.

FIG. 9. (A) Carbon black dye found in canine prostatic acini 24 hours after high-pressure transurethral infusion. (B) Transrectal intraprostatic injection of carbon black demonstrating localized distribution at the site of injection with limited periacinar spread of dye 8 hours after injection. (C) Extracapsular collection of dye following multiple transperineal injection of carbon black utilizing a brachytherapy methodology. H & E. Original magnification×25.

FIG. 10. Photomicrograph of canine prostate after injection of ALVAC-B7 (5×10⁷ pfu total) in fluid-phase (A) and matrix delivered (B) used as a negative control for β-galactosidase activity after staining with X-gal. Representative section of prostate demonstrating β-galactosidase activity after injection of ALVAC-lacZ (5×10⁷ pfu total) delivered in fluid-phase (C) or gelatin sponge matrix (D). Nuclear Fast Red, original magnification×25.

FIG. 11. Photomicrograph of canine prostate following injection of ALVAC-lacZ (5×10⁷ pfu total) with the Gelfoam powder. Improved β-galactosidase gene expression demonstrated over injection of vector in the fluid-phase (A). β-galactosidase activity demonstrated along the prostatic capsule (B). Nuclear Fast Red, original magnification×63.

FIG. 12. Generation of an adenovirus carrying the PSA gene. In order to produce infectious recombinant Ad5 particles recombinant for human PSA, the PSA cDNA was subcloned into the shuttle vector at the Not I and Bam HI sites. This construct was co-transfected with Ad5 μl deletion mutant DNA into HEK 293 cells, where a recombination event occurs placing the PSA gene in the Ad5 genome. The Ad5 E1 gene in the HEK 293 genome provides in trans packaging for the deletion mutant Ad5PSA DNA resulting in the production of infectious by replication-deficient Ad5 particles expressing PSA. The PSA cDNA inserted is the pre-pro form described by Lundwall et al.¹⁸

FIG. 13. Demonstration of the antigen specificity of the anti-PSA CTL. Mice were immunized ip. with 1×10⁹ pfu of virus. On day 14 post-immunization, spleens from each group were pooled and processed. Splenocytes were cultured with stimulator cells for 5 days as described in Materials and Methods. Targets were RM11/neo or RM1/PSA as indicated. Only splenocytes from mice immunized with Ad/PSA can lyse PSA-expressing targets. Data are from 8 experiments with 2-3 mice per group. AdP-RM11/neo=splenocytes from Ad/PSA-immunized mice versus RM11/neo targets; AdZ-RM11/neo splenocytes from Ad/lacZ-immunized mice versus RM11/neo targets; AdP-RM11/PSA=splenocytes from Ad/PSA-immunized mice versus RM1/PSA targets; AdZ-RM11/PSA=splenocytes from Ad/lacZ-immunized mice versus RM1/PSA targets.

FIG. 14. Effect of varying Ad/PSA doses on CTL activity. Mice were immunized ip. with indicated pfu of virus. On day 14 post-immunization, spleens from each group were pooled and processed. Splenocytes were cultured with stimulator cells for 5 days as described in materials and methods. Targets were RM1/PSA. Lytic activity of all groups vs. RM11/neo was not above background and is not shown for clarity. Data are from 4 experiments with 2-3 mice per group. AdP=Ad/PSA; AdZ=Ad/lacZ.

FIG. 15. Duration of CTL response generated by Ad/PSA. Mice were immunized ip. with 1×10⁹ pfu of virus ahead of the harvest date as indicated. Spleens from each group were pooled, processed, and cultured with stimulator cells for 5 days as described in materials and methods. Week 26 data is from a separate harvest. Targets were RM11/PSA. Lytic activity of all groups vs. RM11/neo was not above background and is not shown for clarity. Data are from 3 experiments of 2-3 mice per group. AdP=Ad/PSA; AdZ=Ad/lacZ.

FIG. 16. Identification of the T cell subset mediating PSA-specific lytic activity. Mice were immunized ip. with 1×10⁹ pfu of virus. On days 11, 12, and 13 post-immunization, 100 μg of control or depleting antibody in a volume of 100 μl was injected ip. On day 14 post-immunization, spleens from each group were pooled, processed, and cultured with stimulator cells for 5 days as described in materials and methods. Mice depleted of CD8⁺ T cells cannot lyse RM11/PSA targets. Lytic activity of all groups vs. RM11/neo was not above background and is not shown for clarity. Data are from 2 experiments of 3 mice per group. AdP control antibody=Ad/PSA-immunized mice injected with control antibody SFR8-B6; AdP-CD8=Ad/PSA-immunized mice injected with CD8-depleting antibody 2.43; AdP-CD4=Ad/PSA-immunized mice injected with CD4-depleting antibody GK1.5.

AdP-CD4&8=Ad/PSA-immunized mice injected with both CD4- and CD8-depleting antibodies; AdZ=Ad/lacZ-immunized animals.

FIG. 17. Flow cytometric data for depletion studies. Splenocytes were harvested and labeled as described in materials and methods. Over 99.5% depletion of the desired T cell subsets was achieved. A. Depletion of CD8+ T cells. B. Depletion of CD4+ T cells. Solid lines show data from depleted animals, dashed lines are mice treated with control antibody.

FIG. 18. Splenocyte proliferation in response to 10 μg/ml exogenous PSA. Mice were immunized ip. with 1×10⁹ pfu of virus. On day 14 post-immunization, spleens from each group were pooled, processed, and placed in 96-well plates+/−PSA as described in materials and methods. After 4 days, each well was pulsed with 1 μCi ³H-thymidine for 6 hours and harvested for counting as described in materials and methods. Data are reported as CPM+/−0 standard deviation. Only splenocytes from Ad/PSA-immunized animals proliferated in response to exogenous PSA. Data are from 4 experiments of 2-3 mice per group. PBS=virus diluent control; AdZ=Ad/lacZ-immunized animals; AdP=Ad/PSA-immunized animals.

FIG. 19. Effect of Ad/PSA immunization on RM11/PSA growth. A. Mice were immunized with 1×10⁹ pfu of indicated virus. After 14 days they were challenged sc. on the back with 1×10⁵ RM11/neo or RM11/PSA as indicated. Tumor volumes were calculated once weekly by multiplying length, width, and height. Data are from two experiments with 5 animals per group and are reported as mean tumor volume+/−standard deviation. Control mice were sacrificed at 3 weeks due to large tumor growth. B. Survival data. AdZ-RM11/neo=Ad/lacZ-immunized animals challenged with RM11/neo; AdZ-RM11/PSA=Ad/lacZ-immunized animals challenged with RM11/PSA; AdP-RM11/neo=Ad/PSA-immunized animals challenged with RM11/neo; AdP-RM11/PSA=Ad/PSA immunized animals challenged with RM11/PSA.

FIG. 20. Identification of the effector cell population mediating protection after Ad/PSA immunization. Mice were immunized ip. with 1×10⁹ pfu of virus. On days 11, 12, and 13, 100 μg of control or depleting antibody in a volume of 100 μl was injected ip., and every other day thereafter. On day 14, mice were challenged sc. on the back with 1×10⁵ RM11/PSA tumor cells. Tumor volumes were calculated once weekly by multiplying length, width, and height. Data are from 2 experiments of 5 mice per group and are reported as mean tumor volume+/−standard deviation. Except for AdP-control vs. AdP-NK, all 3 week data values are statistically significant from each other (p<0.05) as determined by the two-tailed Wilcoxon signed-rank test. AdP-control=Ad/PSA-immunized mice injected with control antibody SFR8-B6; AdP-CD8=Ad/PSA-immunized mice injected with CD8-depleting antibody 2.43; AdP-CD4=Ad/PSA-immunized mice injected with CD4-depleting antibody GK1.5; AdP-NK=Ad/PSA-immunized mice with the NK cell-depleting antibody anti-asialo GM1; AdZ=Ad/lacZ-immunized animals.

FIG. 21. Destruction of Established Tumors. A. Mice were injected sc. with 1×10⁵ RM11/PSA clone E6 cells, and after 3 days, were immunized ip. with 1×10⁹ pfu of either Ad/PSA or Ad/lacZ. Seven days after adenovirus immunization, mice were given intra-tumoral injections of 3×10⁷ pfu ALVAC parental virus, 1×10⁷ pfu each of ALVAC-TNF-α, -IL-12, and -IL-2, or matrix alone (day 0). Data is representative of two experiments of 5-6 animals per group. Tumor measurements were discontinued at day 14 when control animals had to be euthanized for humane reasons. B. Combined survival data. All mice at day 40 are tumor-free. AdZ/no RX=mice immunized with Ad/lacZ; AdZ/parent=mice immunized with Ad/lacZ and injected with parental ALVAC vector; AdZ/cyto=mice immunized with Ad/lacZ and injected with ALVAC cytokine vectors; AdP/no RX=mice immumized with Ad/PSA; AdP/parent=mice immunized with Ad/PSA and injected with parental ALVAC vector; AdP/cyto=mice immunized with Ad/PSA and injected with ALVAC cytokine vectors.

FIG. 22. A graph comparing Ad/PSA immunization when delivered by the Collagen matrix composition of the invention and without the collgen system. (E9=10⁹ pfu virus).

FIG. 23. A graph comparing immunization in the presence of antibody to adenovirus where the immunization is delivered via the collagen carrier per the methods of the invention and via fluid phase without a collagen carrier. The collagen matrix provided for significantly increased immunization.

FIG. 24. A graph comparing the immunogen ovalbumin when delivered by fluid phase and with the collagen carrier. The results indicate that the Ad/ova administered by the collagen carrier was a much better immunogen than fluid phase adenovirus where the immunizing antigen is an autologous protein.

FIG. 25. A graph comparing Gelfoam and aqueous feliver showing that Gelfoam augments adenovirus mediated gene delivery.

FIG. 26. A graph showing a comparison of the effects of Gelfoam on gene delivery with either ALVAC (canarypox virus) and type 5 non-replicative Adenovirus.

FIG. 27. A graph showing time course of expression with Gelfoam for both ALVAC and Adenovirus

FIG. 28. A graph demonstrating the effect of Gelfoam on the antitumor activity of ALVAC carrying the genes for IL-2, IL12 and TNF. Tumor target is RM-1, a prostate cancer cell line. Only virus administered with Gelfoam resulted in effective antitumor activity (complete inhibition of tumor outgrowth in 40% of mice).

FIG. 29. A graph showing the determination that CD4+ and CD8+ T cells mediate the antitumor effects induced by combination Gelfoam and ALVAC-IL2,IL12,TNF treatment.

FIG. 30. A graph showing independent confirmation that Gelfoam augments T cell activation. Mice were immunized with recombinant Adenovirus carrying the gene for ovalbumin (Ad5-mOVA), a common antigen used to measure T cell activation. In this study varying doses of Ad5-mOVA were injected in aqueous phase or with Gelfoam. Effective lytic activity was observed at lower virus doses with Gelfoam.

FIG. 31. A graph depicting that Gelfoam has the ability to overcome the inhibitory effects of circulating antibody to Adenovirus on T cell activation. Mice were preimmunized with parental non-replicative adenovirus. Once antibody concentrations to adenovirus were confirmed, mice were immunized with Ad5-mOVA with or without Gelfoam. When Gelfoam was included, no inhibitory effects on T cell activation were observed.

FIG. 32. A chart showing a comparison of the gene delivery capacity of aquaeous phase, Gelfoam, and DeFlux to augment gene delivery. Deflux is a combination of cross-linked dextran and hyaluronidase. The non-collagen based matrix augments gene delivery at a level equivalent to that of Gelfoam FIG. 33. A graph showing that while Deflux also augments gene delivery, it does not enhance T cell activation. Only Gelfoam augments both gene delivery and T cell activation.

FIG. 34. A graph shwoing the results of experiments that tested the ability of Gelfoam to augment gene delivery in CD154 knockout mice. CD154 is an important TNF family member that is known to augment T cell responses and induce isotype switching in B cell responses. The data show that although gene expression is lower in CD 154 knockout mice, Gelfoam still augments gene delivery. The lower gene expression in CD 154 mice is consistent in many experiments, suggesting that this ligand is important in gene delivery.

FIG. 35. A graph shwoing the results of experiments that tested the ability of Gelfoam to augment T cell activation in CD154 knockout mice. Gelfoam lost its ability to enhance T cell activation in these mice (CD 154 knockouts).

FIG. 36. A graph shwoing the results of experiments that tested the ability of Gelfoam to augment gene delivery in genetically modified mice, including CD 154−/−, FasL−/−, and double knockout mice (CD154−/− & FasL−/−). Note some loss of gene expression in single knockout mice but complete loss in double knockout mice.

### DETAILED DESCRIPTION OF THE INVENTION

Collagen is the major protein (comprising over half of that in mammals) of the white fibers of connective tissue, cartilage, and bone, that is insoluble in water but can be altered to easily digestible, soluble gelatins by boiling in water, dilute acids, or alkalies. It is high in glycine, L-alanine, L-proline, and L-4-hydroxyproline, but is low in sulfur and has no L-tryptophan. Collagen comprises a large family of genetically distinct molecules all of which have a unique triple helix configuration of three polypeptide subunits; 19 types of collagen have been identified in vertebrates. See Brown, J. C. et al, “The collagen Superfamily”, Int. Arch. Allergy Immunol. 1995 August; 107 (4):484-90. Any of these types of collagen may be used for the invention. The term “collagen” as used herein shall not be limited to any particular form or type of collagen but shall include all types as described and disclosed herein.

Another particular example of a suitable material is fibrous collagen, which may be lyophilized following extraction and partial purification from tissue and then sterilized. Matrices may also be prepared from tendon or dermal collagen, as may be obtained from a variety of commercial sources, such as, eg., Sigma and Collagen Corporation. Collagen matrices may also be prepared as described in U.S. Pat. Nos. 4,394,370 and 4,975,527, each incorporated herein by reference.

In addition, lattices made of collagen and glycosaminoglycan (GAG) such as that described in Yannas & Burke, U.S. Pat. No. 4,505,266, may be used in the practice of the invention. The collagen/GAG matrix may effectively serve as a support or “scaffolding” structure into which repair cells may migrate. Collagen matrix such as those disclosed in Bell, U.S. Pat. No. 4,485,097, may also be used as a matrix material.

The various collagenous materials may also be in the form of mineralized collagen. For example, the fibrous collagen implant material termed UltraFiber™, as may be obtained from Norian Corp., (1025 Terra Bella Ave., Mountain View, Calif., 94043) may be used for formation of matrices. U.S. Pat. No. 5,231,169, incorporated herein by reference, describes the preparation of mineralized collagen through the formation of calcium phosphate mineral under mild agitation in situ in the presence of dispersed collagen fibrils. Such a formulation may be employed in the context of delivering a nucleic acid segment to a bone tissue site. Mineralized collagen may be employed, for example, as part of gene activated matrix therapeutic kit for fracture repair.

At least 20 different forms of collagen have been identified and each of these collagens may be used in the practice of the invention. For example, collagen may be purified from hyaline cartilage, as isolated from diarthrodial joints or growth plates. Type II collagen purified from hyaline cartilage is commercially available and may be purchased from, e.g., Sigma Chemical Company, St. Louis. Type I collagen from rat tail tendon may be purchased from, e.g., Collagen Corporation. Any form of recombinant collagen may also be employed, as may be obtained from a collagen-expressing recombinant host cell, including bacterial yeast, mammalian, and insect cells. When using collagen as a matrix material it may be advantageous to remove what is referred to as the “telopeptide” which is located at the end of the collagen molecule and known to induce an inflammatory response.

The collagen used in the invention may, if desired be supplemented with additional minerals, such as calcium, e.g., in the form of calcium phosphate. Both native and recombinant type collagen may be supplemented by admixing, absorbing, or otherwise associating with, additional minerals in this manner.

The collagen may also be in the form of a matrix, although powdered non-cross-linked collagen has also been shown to be useful for the invention. A number of collagen based matrix compositions are commercially available, such as Gelfoam® which is commercially available through Pharmacia & Upjohn, Kalamazoo, Mich. Gelfoam® is an absorbable gelatin sterile hemostatic agent prepared from purified skin gelatin. Gelfoam is thought to work by releasing thromboplastin from platelets. It interacts with prothrombin and calcium to produce thrombin which initiates the clotting reaction.

While not wishing to be bound by any theory it is postulated that the collagen carrier composition serves to increase nucleotide expression by mobilizing factors associated with the clotting response that provide for increased expression of the nucleotide expression system.

The carrier composition may become impregnated with the nucleotide expression system simply by soaking the carrier composition in a solution containing the polynucleotide, for a brief time anywhere from five minutes up to an hour or longer.

The carrier composition may become impregnated with the nucleotide expression system simply by soaking the collagen carrier composition in a solution containing the polynucleotide, for a brief time anywhere from five minutes up to an hour or longer.

The expression system of the invention in its simplest context comprises an expression system including a therapeutic gene or nucleotide sequence the expression of which is desired in a recipient cell and a collagen carrier. Any nucleotide sequence as defined supra can be used in the expression system of the invention.

In a preferred embodiment the expression system is one which is designed to inhibit the growth of or destroy neoplastic cells. This can include therapeutic genes such as a suicide gene, for example, the Herpes Simplex Virus thymidine kinase gene, which upon treatment with ganciclovir destroys all transduced cells expressing the gene, or an immune stimulating gene such as tissue necrosis factor or interleukins which stimulate the immune response of the host to kill neoplastic cells, as described in one or more of the following:
Crouzet et al., WO 97FR193 (Aug. 14, 1997) “Nucleic acid encoding mutant thymidine-kinase and related proteins and vectors—herpes simplex virus recombinant enzyme production and DNA application in cancer or restenosis gene therapy”;
Kayyem et al., WO 9611712 (Apr. 25, 1996) “Vehicles for delivering nucleic acid or therapeutic agents to cells—herpes virus thymidine-kinase or antisense DNA expression in cell using an oppositely charged polymer delivery vehicle, for application in cancer gene therapy, and in magnetic resonance imaging”;
Castleden S et al., Br. J. Cancer, 71 (24):13 1995, “The use of combination gene therapies for the treatment of cancer—herpes simplex virus thymidine-kinase and interleukin-2 retro virus vector-mediated expression in mouse lung and spleen tumor metastases and treatment with ganciclovir” (conference abstract);
Chen S et al., Proc. Natl. Acad. Sci. U.S.A., 92 (7):2577-81 1995, “Combination gene therapy for liver metastasis of colon carcinoma in vivo interleukin-2 cytokine-mediated gene therapy using an adeno virus vector”;
Culver K W, Abstr. Pap. Am. Chem. Soc. (208 Meet., Pt. 1, MEDI142) 1994, “Destruction of solid tumors with the herpes thymidine-kinase gene—herpes simplex virus gene application in brain cancer gene therapy” (conference abstract);
Garver Jr R I et al., Gene Ther. 1 (1):46-50 1994, “Strategy for achieving selective killing of carcinomas—use of secretory leukoprotease-inhibitor for toxin targeting to SLPI-expressing carcinoma for gene therapy”.

The nucleotide expression system of the invention and carrier as well as methods of the invention employing genetic engineering techniques using this system have the benefit of increasing survival time of the vector. Virtually all conventional gene therapy protocols can benefit from increased survival time of the therapeutic gene and would therefore benefit by inclusion of this carrier. Similarly, expression systems delivered by the methods and compositions of the invention also exhibit the unexpected result of quantitatively higher expression levels compared to those delivered without the carrier.

In a preferred embodiment the nucleotide expression system of the invention is included within an appropriate gene transfer vehicle which is then used to transduce cells to express the gene of interest and increase half life of the expression system in the recipient host cells. The gene delivery vehicle can be any delivery vehicle known in the art and can include simply naked DNA which is facilitated by a receptor mediated transfection as well as any of a number of vectors. Such vectors include but are not limited to eukaryotic vectors, prokaryotic vectors (such as for example bacterial vectors) and viral vectors including but not limited to retroviral vectors, adenoviral vectors, adeno-associated viral vectors, lentivirus vectors (human and other including porcine), Herpes virus vectors, Epstein-Barr virus vectors, SV40 virus vectors, pox virus vectors, pseudotype virus vectors.

The invention is particularly suited to use of vectors such as retroviral, adeno-associated, or lenti viral vectors which integrate into host DNA. The invention is also particularly suited to vector systems which persist indefinitely in transformed cells such as HSV vectors or those which are maintained in episomal state such as by use of Epstein Barr genetic elements.

Any of a number of standard gene delivery transformation methods can be used such as lipid mediated transfection, receptor mediated transfection, calcium phosphate transfection, electroporation particle bombardment, naked-direct DNA injection, diethylaminoethyl (DEAE-dextran transfection).

The expression vehicles (vectors) of the invention can be engineered by any of a number of techniques known to those of skill in the art. The following is a summary of techniques for construction and transformation of the vectors of the invention.

Genetic Engineering Techniques for Construction and Delivery of Vectors

Any of a number of standard gene delivery transformation methods can be used for the invention including lipid mediated transfection, receptor mediated transfection, calcium phosphate transfection, electroporation particle bombardment, naked-direct DNA injection, diethylaminoethyl (DEAE-dextran transfection).

In a preferred embodiment the expression vehicles or vectors of the invention comprising the expression system also comprise a selectable marker gene to select for transformants as well as a method for selecting those transformants for propagation of the construct in bacteria. Such selectable marker may contain an antibiotic resistance gene, such as those that confer resistance to ampicillin, kanamycin, tetracycline, or streptomycin and the like. These can include genes from prokaryotic or eukaryotic cells such as dihydrofolate reductase or multi-drug resistance I gene, hygromycin B resistance that provide for positive selection. Any type of positive selector marker can be used such as neomycin or Zeosyn and these types of selectors are generally known in the art. Several procedures for insertion and deletion of genes are known to those of skill in the art and are disclosed. For example in Maniantis, “Molecular Cloning”, Cold Spring Harbor Press. See also Post et al., Cell, Vol. 24:555-565 (1981). An entire expression system must be provided for the selectable marker genes and the genes must be flanked on one end or the other with promoter regulatory region and on the other with transcription termination signal (polyadenylation cite). Any known promoter/transcription termination combination can be used with the selectable marker genes. For example SV40 promoter and SV40 poly A.

A therapeutic gene to be expressed can then be introduced into the vector of the invention. The foreign DNA can comprise an entire transcription unit, promoter-gene-poly A or the vector can be engineered to contain promoter/transcription termination sequences such that only the gene of interest need be inserted. These types of control sequences are known in the art and include promoters for transcription initiation, optionally with an operator along with ribosome binding site sequences. Examples of such systems include beta-lactase (penicillinase) and lactose promoter systems, (Chang et al., Nature, 1977, 198:1056); the Tryptophan (trp) promoter system (Goeddel, et al., Nucleic Acid Res., 1980, 8:4057) and the lambda derived P1 promoter and N-gene ribosome binding site (Shimatake et al., Nature 1981, 292:128). Other promoters such as cytomegalovirus promoter or Rous Sarcoma Virus can be used in combination with various ribosome elements such as SV40 poly A. The promoter can be any promoter known in the art including constitutive, (supra) inducible, (tetracycline-controlled transactivator (tTA)-responsive promoter (tet system, Paulus, W. et al., “Self-Contained, Tetracycline-Regulated Retroviral Vector System for Gene Delivery to Mammalian Cells”, J of Virology, January 1996, Vol. 70, No. 1, pp. 62-67)), or tissue specific, (such as those cited in Costa, et. Al., European journal of Biochemistry, 258 “Transcriptional Regulation Of The Tissue-Type Plasminogen Activator Gene In Human Endothelial Cells: Identification Of Nuclear Factors That Recognize Functional Elements In The Tissue-Type Plasminogen Activator Gene Promoter” pgs, 123-131 (1998); Fleischmann, M., et. Al., FEBS Letters 440 “Cardiac Specific Expression Of The Green Fluorescent Protein During Early Murine Embryonic Development” pgs. 370-376, (1998); Fassati, Ariberto, et. Al., Human Gene Therapy, (9:2459-2468) “Insertion Of Two Independent Enhancers In The Long Terminal Repeat Of A Self Inactivating Vector Results In High-Titer Retroviral Vectors With Tissue Specific Expression” (1998); Valerie, Jerome, et. Al. Human Gene Therapy 9:2653-2659, “Tissue Specific Cell Cycle Regulated Chimeric Transcription Factors For The Targeting Of Gene Expression To Tumor Cells, (1998); Takehito, Igarashi, et. Al., Human Gene Therapy 9:2691-2698, “A Novel Strategy Of Cell Targeting Based On Tissue-Specific Expression Of The Ecotropic Retrovirus Receptor Gene”, 1998; Lidberg, Ulf et. al. The Journal of Biological Chemistry 273, No. 47, “Transcriptional Regulation Of The Human Carboxyl Ester Lipase Gene In Exocrine Pancreas” 1998; Yu, Geng-Sheng et. Al., The Journal of Biological Chemistry 273 No. 49, “Co-Regulation Of Tissue-Specific Alternative Human Carnitine Palmitoyltransferase IB Gene Promoters By Fatty Acid Enzyme Substrate” (1998)). These types of sequences are well known in the art and are commercially available through several sources, ATCC, Pharmacia, Invitrogen, Stratagene, Promega.

In a most preferred embodiment the vector comprises a specifically engineered multi-cloning site within which several unique restriction sites are created. Restriction enzymes and their cleavage sites are well known to those of skill in the art.

In a preferred embodiment, a packaging cell line is transduced with a viral vector containing the therapeutic nucleotide sequence to form a producer cell line including the viral vector. The producer cells may then be directly administered, whereby the producer cells generate viral particles capable of transducing the recipient cells.

In a preferred embodiment, the viral vector is a retroviral vector. Examples of retroviral vectors which may be employed include, but are not limited to, Moloney Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus.

Retroviral vectors are useful as agents to mediate retroviral-mediated gene transfer into eukaryotic cells. Retroviral vectors are generally constructed such that the majority of sequences coding for the structural genes of the virus are deleted and replaced by the therapeutic gene(s) of interest. Most often, the structural genes (i.e., gag, pol, and env), are removed from the retroviral backbone using genetic engineering techniques known in the art. This may include digestion with the appropriate restriction endonuclease or, in some instances, with Bal 31 exonuclease to generate fragments containing appropriate portions of the packaging signal.

These new genes may be incorporated into the proviral backbone in several general ways. The most straightforward constructions are ones in which the structural genes of the retrovirus are replaced by a single gene which then is transcribed under the control of the viral regulatory sequences within the long terminal repeat (LTR). Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

Efforts have been directed at minimizing the viral component of the viral backbone, largely in an effort to reduce the chance for recombination between the vector and the packaging-defective helper virus within packaging cells. A packaging-defective helper virus is necessary to provide the structural genes of a retrovirus, which have been deleted from the vector itself.

In one embodiment, the retroviral vector may be one of a series of vectors described in Bender, et al., J. Virol. 61:1639-1649 (1987), based on the N2 vector (Armentano, et al., J. Virol., 61:1647-1650) containing a series of deletions and substitutions to reduce to an absolute minimum the homology between the vector and packaging systems. These changes have also reduced the likelihood that viral proteins would be expressed. In the first of these vectors, LNL-XHC, there was altered, by site-directed mutagenesis, the natural ATG start codon of gag to TAG, thereby eliminating unintended protein synthesis from that point.

In Moloney murine leukemia virus (MoMuLV), 5′ to the authentic gag start, an open reading frame exists which permits expression of another glycosylated protein (pPr80^{gag}). Moloney murine sarcoma virus (MoMuSV) has alterations in this 5′ region, including a frameshift and loss of glycosylation sites, which obviate potential expression of the amino terminus of pPr80^{gag}. Therefore, the vector LNL6 was made, which incorporated both the altered ATG of LNL-XHC and the 5′ portion of MoMuSV. The 5′ structure of the LN vector series thus eliminates the possibility of expression of retroviral reading frames, with the subsequent production of viral antigens in genetically transduced target cells. In a final alteration to reduce overlap with packaging-defective helper virus, Miller has eliminated extra env sequences immediately preceding the 3′ LTR in the LN vector (Miller, et al., Biotechniques, 7:980-990, 1989).

The paramount need that must be satisfied by any gene transfer system for its application to gene therapy is safety. Safety is derived from the combination of vector genome structure together with the packaging system that is utilized for production of the infectious vector. Miller, et al. have developed the combination of the pPAM3 plasmid (the packaging-defective helper genome) for expression of retroviral structural proteins together with the LN vector series to make a vector packaging system where the generation of recombinant wild-type retrovirus is reduced to a minimum through the elimination of nearly all sites of recombination between the vector genome and the packaging-defective helper genome (i.e. LN with pPAM3).

In one embodiment, the retroviral vector may be a Moloney Murine Leukemia Virus of the LN series of vectors, such as those hereinabove mentioned, and described further in Bender, et al. (1987) and Miller, et al. (1989). Such vectors have a portion of the packaging signal derived from a mouse sarcoma virus, and a mutated gag initiation codon. The term “mutated” as used herein means that the gag initiation codon has been deleted or altered such that the gag protein or fragment or truncations thereof, are not expressed.

In another embodiment, the retroviral vector may include at least four cloning, or restriction enzyme recognition sites, wherein at least two of the sites have an average frequency of appearance in eukaryotic genes of less than once in 10,000 base pairs; i.e., the restriction product has an average DNA size of at least 10,000 base pairs. Preferred cloning sites are selected from the group consisting of NotI, SnaBI, SalI, and XhoI. In a preferred embodiment, the retroviral vector includes each of these cloning sites.

When a retroviral vector including such cloning sites is employed, there may also be provided a shuttle cloning vector which includes at least two cloning sites which are compatible with at least two cloning sites selected from the group consisting of NotI, SnaBI, SalI, and XhoI located on the retroviral vector. The shuttle cloning vector also includes at least one desired gene which is capable of being transferred from the shuttle cloning vector to the retroviral vector.

The shuttle cloning vector may be constructed from a basic “backbone” vector or fragment to which are ligated one or more linkers which include cloning or restriction enzyme recognition sites. Included in the cloning sites are the compatible, or complementary cloning sites hereinabove described. Genes and/or promoters having ends corresponding to the restriction sites of the shuttle vector may be ligated into the shuttle vector through techniques known in the art.

The shuttle cloning vector can be employed to amplify DNA sequences in prokaryotic systems. The shuttle cloning vector may be prepared from plasmids generally used in prokaryotic systems and in particular in bacteria. Thus, for example, the shuttle cloning vector may be derived from plasmids such as pBR322; pUC 18; etc.

The vector includes one or more promoters. Suitable promoters which may be employed include, but are not limited to, the retroviral LTR; the SV40 promoter; and the human cytomegalovirus (CMV) promoter described in Miller, et al., Biotechniques, 7:(9):980-990 (1989), or any other promoter (e.g., cellular promoters such as eukaryotic cellular promoters including, but not limited to, the histone, pol III, and β-actin promoters). Other viral promoters which may be employed include, but are not limited to, adenovirus promoters, TK promoters, and B 19 parvovirus promoters. The selection of a suitable promoter will be apparent to those skilled in the art from the teachings contained herein.

The vector then is employed to transduce a packaging cell line to form a producer cell line. Examples of packaging cells which may be transfected include, but are not limited to the PE501, PA317, T2, T-AM, PA12, T19-14×, VT-19-17-H2, ACRE, TCRIP, GP+E-86, GP+envAM12, and DAN cell lines. The vector containing the therapeutic nucleic acid sequence may transduce the packaging cells through any means known in the art. Such means include, but are not limited to, electroporation, the use of liposomes, and CaPO₄ precipitation.

The producer cells then are administered directly to or adjacent to desired recipient cells.

In a preferred embodiment the invention comprises a viral vector which commonly infects humans and packaging cell line which is human based. For example vectors derived from viruses which commonly infect humans such as Herpes Virus, Epstein Barr Virus, may be used which do not express an active α-galactosyl envelope.

In yet another embodiment the vector comprises a Herpes Simplex Virus plasmid vector. Herpes simplex virus type-1 (HSV-1) has been demonstrated as a potential useful gene delivery vector system for gene therapy, Glorioso, J. C., “Development of Herpes Simplex Virus Vectors for Gene Transfer to the Central Nervous System. Gene Therapeutics: Methods and Applications of Direct Gene Transfer”, Jon A. Wolff, Editor, 1994 Birkhauser Boston, 281-302; Kennedy, P. G., “The Use of Herpes Simplex Virus Vectors for Gene Therapy in Neurological Diseases”, Q J Med, November 1993, 86 (11):697-702; Latchman, D. S., “Herpes Simplex Virus Vectors for Gene Therapy”, Mol Biotechnol, October 1994, 2 (2):179-95.

HSV-1 vectors have been used for transfer of genes to muscle. Huard, J., “Herpes Simplex Virus Type 1 Vector Mediated Gene Transfer to Muscle”, Gene Therapy, 1995, 2, 385-392; and brain, Kaplitt, M. G., “Preproenkephalin Promoter Yields Region-Specific and Long-Term Expression in Adult Brain After Direct In Vivo Gene Transfer Via a Defective Herpes Simplex Viral Vector”, Proc Natl Acad Sci USA, Sep. 13, 1994, 91 (19):8979-83, and have been used for murine brain tumor treatment, Boviatsis, E. J., “Long-Term Survival of Rats Harboring Brain Neoplasms Treated With Ganciclovir and a Herpes Simplex Virus Vector That Retains an Intact Thymidine Kinase Gene”, Cancer Res, Nov. 15, 1994, 54 (22):5745-51; Mineta, T., “Treatment of Malignant Gliomas Using Ganciclovir-Hypersensitive, Ribonucleotide Reductase-Deficient Herpes Simplex Viral Mutant”, Cancer Res, Aug. 1, 1994, 54 (15):3963-6.

Helper virus dependent mini-viral vectors have been developed for easier operation and their capacity for larger insertion (up to 140 kb), Geller, Al, “An Efficient Deletion Mutant Packaging System for Defective Herpes Simplex Virus Vectors: Potential Applications to Human Gene Therapy and Neuronal Physiology”, Proc Natl Acad Sci USA, November 1990, 87 (22):8950-4; Frenkel, N., “The Herpes Simplex Virus Amplicon: A Versatile Defective Virus Vector”, Gene Therapy. 1. Supplement 1, 1994. Replication incompetent HSV amplicons have been constructed in the art, one example is the pHSVlac vector by Geller et al, Science, 241, September 1988, incorporated herein by reference. These HSV amplicons contain large deletions of the HSV genome to provide space for insertion of exogenous DNA. Typically they comprise the HSV-1 packaging site, the HSV-1 “ori S” replication site and the IE 4/5 promoter sequence. These virions are dependent on a helper virus for propagation.

Primarily two types of mutant helper viruses have been developed to minimize recombination. Other complementary HSV helper virus systems are contemplated herein and are within the scope of those of skill in the art. One such system which has been developed is a temperature-sensitive mutant. An HSV temperature-sensitive (TS) mutant has been developed with a TS mutation in the IE3 gene. Davison et al, 1984, J. Gen. Virol., 65:859-863. Consequently this virus has an IE phenotype, does not replicate DNA, does not significantly alter cellular physiology, and does not produce progeny virus at 37° C. Virus is grown at the permissive temperature of 37° C. TS mutants however have had a tendency to revert to wild type.

In contrast a second helper virus system is a deletion mutant with the majority of the IE3 gene simply deleted. These do not revert to wild type. Therefore HSV-1 vectors packaged using a deletion mutant as helper virus is the most preferred helper virus of the invention. See for example Patterson et al., 1990, J. Gen. Virol., 71:1775-1783. Other replication incompetent helper viruses can be used and one of skill in the art will appreciate that other mutations in the IE genes or other genes which result in a replication incompetent helper virus which will provide the appropriate replication and expression functions and which are coordinated with the helper cell line and vector are contemplated within this invention. Any cell line can be used for this step so long as it is capable of expressing the IE3 or replication dependent gene, or obtaining a helper cell line which has already been transformed and is commercially available. Any cell line can be used by introducing pHE and the plasmid containing the IE3 gene simultaneously. Next, the vector is delivered to the helper cell line by electroporation, calcium phosphate DNA transfection or any other suitable method. Any cell line can be used by introducing pHE and the plasmid containing the IE3 gene simultaneously. The cells are next infected with a helper virus IE3 deletion mutant or other corresponding deletion mutant which is replication incompetent. The IE3 gene or other such gene in the helper cell line complements the helper virus resulting in a productive HSV-1 infection and the resulting virus stock consists of HSV-1 particles containing either vector DNA or helper virus DNA, all of which are replication incompetent. Further information about helper cell lines and the methodology is disclosed in Geller et al., PNAS, 87:8950-8954, November 1990, “An Efficient Deletion Mutant Packaging System for Defective Herpes Simplex Virus Vectors: Potential Applications to Human Gene Therapy and Neuronal Physiology”. The invention comprises a HSV mini vector which combines a replication incompetent HSV amplicon with other viral sequences such as those from Epstein-Barr virus, human papillomavirus, or bovine papillomavirus type 1 which allow the vector to be maintained in the cell in episomal form achieving a 10 times greater titer, and a very large DNA insert capacity.

One embodiment of the present invention involves a helper virus-dependent mini-viral vector comprising: (a) the HSV-1 “a” sequence for the package/cleavage signal and an “ori S” replication origin for the replication packaging of the plasmid (in response to signals to replicate and package from the helper virus); (b) an Epstein-Barr virus (EBV) nuclear antigen (EBNA-1) gene and an EBV latent origin of replication (ori P) which allow the vector to be maintained in episomal form within the nucleus for replication without integration to the host genome and for even replication into each of two dividing cells; preferably (c) genes from prokaryotic cells for propagation of the vector in E. coli (a selectable marker gene such as the ampicillin resistance or tetracycline resistance gene and the col. E1 ori) and (d) a sequence encoding a immune suppression protein such as US11. Optionally the vector may also comprise prokaryotic genes that provide for a second selectable marker such as the genes for positive Hygromycin selection.

In this particular embodiment the packaging function of mini-vector DNA into Herpes simplex viral capsids is provided by a helper virus and a helper cell line.

In yet another embodiment the HSV vector can be engineered to produce a helper free viral vector as in Mann et al., “Construction of a Retro-Virus Packaging Mutant and its Use to Produce Helper-Free Defective Retrovirus”, 33 Sal., p. 153-159, May 1983, Journal of Virology, September 1989, pp. 3822-3829, September 1989; Samulski “Helper Free Stocks of Recombinant Adeno-Associated Viruses: Normal Integration Does Not Require Viral Gene Expression”; and Kohn et al., “High Efficiency Gene Transfer Into Mammalian Cells: Generation of Helper-Free Recombinant Retrovirus With Broad Mammalian Host Range”, PNAS, 81:6349-6353, October 1984. See also Okasinki, U.S. Pat. No. 4,970,155 “HSV HELPER VIRUS INDEPENDENT VECTOR”, incorporated herein by reference.

The expression system delivery composition of the present invention can be used for any diagnostic or therapeutic genetic engineering protocol including in vitro, ex vivo, or in vivo expression of a desired nucleotide sequence. For example the expression vehicles of the invention can be used in any of a number of therapeutic treatment protocols in the treatment of cancer such as by the Herpes simplex virus, thymidine kinase gene transfer system Martuza R L et al., “Experimental therapy of human glioma by means of a genetically engineered virus mutant”, Science, 1991; 252:854-856). Also in ex vivo gene therapy protocols such as bone marrow purging (Seth P., et al., “Adenovirus-mediated gene transfer to human breast tumor cells: an approach for cancer gene therapy and bone marrow purging”, Cancer Res. 56 (6): 1346-¹³⁵I (1996; Andersen, N. S., et al., “Failure of immunologic purging in mantle cell lymphoma assessed by polymerase chain reaction detection of minimal residual disease”, Blood, 90 (10:4212-4221 (1997)) thus when the transformed cells are reintroduced to the patient they will generate a decreased immune response. These may also be used for diagnostic purposes as well.

To fully exploit the benefits of the methods and compositions described herein, the use of many general gene therapy improvements are contemplated and are intended to be within the scope of this invention. In this manner, improvements as higher viral titer production, selection of therapeutic gene and promotor enhancer elements will be utilized, and are intended to be within the scope of the invention. These improvements are seen as simply characterized through routine experimentation and are intended to be within the scope of this invention.

All references cited herein are hereby expressly incorporated in their entirety be reference including the following:
Miller, et al., “Redesign of retrovirus packaging cell lines to avoid recombination leading to helper virus production”, Molecular and Cellular Biology, 6:(8), 2895-2902 (1986)
Koenig, Scott, “A lesson from the HIV patient: The immune response is still the bane (or promise) of gene therapy”, Nature Medicine, 2:(6), 165 (1996)
York, et al., “A cytosolic herpes simplex virus protein inhibits antigen presentation to CD8+ T lymphocytes”, Cell, 11: 525-535 (1994)
Rosenthal, K., “Cells Expressing Herpes Simplex Virus Glycoprotein gC byt not gB, gD, or gE are recognized by murine virus-specific cytotoxic T lymphocytes”, Journal of Virology, 61 (8) 2438-2447 (1987)
Jones, et al., “Multiple independent loci within the human cytomegalovirus unique short region down-regulate expression of major histocompatibility complex Class I heavy chains”, Journal of Virology, 69 (8) 4830-4841
Wiertz, et al., “The human cytomegalovirus US11 gene product dislocates MHC Class 1 heavy chains from the endoplasmic reticulum to the cytosol”, Cell, 84 769-779
Hill, et al., “Herpes simplex virus turns off the TAP to evade host immunity”, Nature, 375 411-415 (1995)
Galocha et al., “The active site of ICP47, a herpes simplex virus-encoded inhibitor of the major histocompatibility complex (MHC)-encoded peptide transporter associated with Antigen Processing (TAP), maps to the NH₂-terminal 35 residues”, 185 (9) 1565-1572 (1997)
Tomazin et al., “Stable binding of the herpes simplex virus ICP47 protein to the peptide binding site of TAP”, EMBO J15 (13) 3256-3266 (1996)
Kwangseog et al., “Molecular mechanism and species specificity of TAP inhibition by herpes simplex virus protein ICP47”, EMBO J. 15 (13) 3247-3255 (1996)
Früh et al., “A viral inhibitor of peptide transporter for antigen presentation”, Nature, 375 415-418 (1995)
Machold et al., “The HCMV gene products US11 and US2 differ in their ability to attack allelic forms of murine major histocompatibility complex (MHC Class I heavy chains”, J Exp. Med., 185 (2) 363-366 (1997)

### Jones et al., “Human cytomegalovirus US2 destabilizes major histocompatibility complex Class I heavy chains”, Journal of Virology, 71 (2) 2970-2979 (1997)

Nagata et al., “Destruction of pancreatic islet cells by cytotoxic T lymphocytes in nonobese diabetic mice”, Journal of Immunology, 143, 1155-1162 (1978)
Qin et al., “Retrovirus-mediated transfer of viral IL-10 gene prolongs murine cardiac allograft survival”, The Journal of Immunology, 156 2316-2323 (1996)
Parks, et al., “Encephalomyocarditis virus 3C protease: efficient cell-free expression from clones which link viral 5′ noncoding sequences to the P3 region”, Journal of Virology 60 (2) 376-384 (1986)
Früh et al., “A viral inhibitor of peptide transporters for antigen presentation”, Nature, 375 415-418 (1995)

In another embodiment, the gene delivery vehicles of the invention have been shown to provide antigen specific protective immunity. Thus the collagen carrier may also be used not only to deliver nucleotide expressions systems but also immunogenic proteins as well as the nucleotide sequences encoding these proteins. This can provide for delivery and any antigenic protein or nucleotide sequence encoding the same for vaccine type protocols. According to the invention priming cells with cancer specific immunizing expression systems which delivered prostate specific antigen provided for protection and elimination of tumors of unprecedented size upon challenge with tumor cells and potentiated any subsequent treatment with cytokines. The collagen carrier composition provided for a specific antibody response that was not observed when the PSA was delivered by other delivery systems. See FIG. 22. This delivery system can thus be used for any antigen to potentiate the immune response that is observed, and with any cytokine to potentiate any elimination of cancer cells. Numerous cancer specific antigens and cytokines are known in the art and are disclosed for example through gen bank and in the references incorporated herein. The following examples are intended to further illustrate the compositions and methods of the invention and are intended to limit the invention in no way.

### EXAMPLES

Animals and Tumor Cells

The murine prostate cancer model, RM-1, was obtained from Dr. Timothy C. Thompson (Baylor College of Medicine, Houston, Tex.). This model mimics multi-step carcinogenesis by activating the ras and myc oncogenes and is used to induce an aggressive prostate carcinoma in vivo. This cell line retains many features of prostate cancer including androgen responsiveness early in culture, expression of androgen receptor, and progression to androgen independence with time. MB-49, a chemically-induced mouse bladder tumor, was used in concert with RM-1 throughout the in vitro and in vivo experiments. Cultured cells were maintained in Dulbeccos' Modified Eagle's Medium (DMEM) containing 10% fetal calf serum (FCS). Both RM-1 and MB-49 are synergistic to C57BL/6 mice.

The mice (6-8 weeks of age at the time of study initiation) were obtained through the National Cancer Institute. Mice were allowed free access to food and water. All animal studies were approved by the University of Iowa Animal Review Board and were performed in accordance with institutional guidelines.

Gene Transfer Vectors

ALVAC is a canarypox virus that can infect mammalian cells, but is restricted to avian species for replication and has been shown to be a safe and effective vector in both humans and animals. The viral strain from which ALVAC was obtained was isolated from a pox lesion on an infected canary. Parental ALVAC, ALVAC vectors encoding murine IL-2, murine IL-12, and murine TNFα, as well as the reporter gene constructs β-galactoside (ALVAC-lacZ), Green Fluorescent Protein (ALVAC-GFP), and luciferase (ALVAC-luciferase) were developed at Virogenetics Corporation (Troy, N.Y.).

Delivery System

Four delivery matrices were compared to determine delivery ability to cells in vitro and in vivo. The substances were chosen. Polyglycolic acid (Davis and Geck, Inc., Wayne, N.J.) and chromic catgut (Ethicon, Inc., Somerville, N.J.) are both absorbable suture material. Plain catgut spacer material (MDTech, Gainsville, Fla.) is a commercially-available product used for prostate cancer brachytherapy protocols. Gelfoam® (Pharmacia and Upjohn, Kalamazoo, Mich.) is an absorbable gelatin sponge prepared from purified pork skin gelatin granules and is used as a hemostatic agent. All delivery systems were prepared in 6 mm lengths and were tested in vitro and in vivo through an 18-gauge spinal needle (Becton Dickinson and Co., Franklin, N.J.) to better mimic intra-prostatic injection in a clinical trial.

As has been previously described in the literature for the carrier delivery of insulin, the virion concentration to be delivered by an individual matrix was determined by weighing the matrices before and after viral absorption. The dry and wet weights of the matrices after one minute of viral absorption were recorded and the subsequent volume delivered was calculated. See Table 1. Subsequently, to ensure that the gelatin sponge matrix was reliably delivering this weight-calculated number of viral particles, comparisons were made utilizing particle determination by the optical density (OD) at 260 nm after digestion of the gelatin matrix. A calculated particle concentration was delivered into solution by the gelatin sponge matrix and then digested by a combination of collagenase (0.16%), bovine serum albumin (2.5%), and DNAase (0.001%) in PBS.

Infection and Reporter Gene Assays

For in vitro analysis, RM-1 or MB-49 cells were harvested and plated with DMEM containing 10% FCS and 10 mM HEPES buffer (pH 7.0) on the day prior to infection. The medium was changed to DMEM with 2% FCS at the time of viral infection with either ALVAC-luciferase or ALVAC-lacZ delivered directly into culture or via the delivery matrices. The viral vectors were added to the cells at the multiplicity of infection (MOI)-plaque forming units per cell shown in each experiment. The cells were then incubated for 6 hours at 37° C. in an atmosphere of 5% CO₂ when the media was changed to DMEM with 10% FCS. Reporter assays were performed at 48 hours after infection and all in vitro experiments were performed in triplicate and repeated in at least two experiments.

For the in vivo studies, RM-1 or MB-49 cells were harvested from the tissue plates by treatment with 10 mM EDTA and were washed with PBS (pH 7.2). The cells were then resuspended in DMEM in a concentration of 5×10⁶, and 0.1 mL was injected subcutaneously into the backs of mice. The ALVAC vectors recombinant for luciferase or β-galactosidase (β-gal) were injected either directly or via the delivery systems at a concentration of 3×10⁶ plaque forming units (pfu) per mL approximately 10 days after tumor implantation. Tumors at this time were approximately 8 mm by 8 mm (˜200 mg wet weight). Tumors were harvested for reporter assays at various times after infection as described for individual experiments. Experiments were performed at least twice for MB-49 as well as the RM-1 tumors in vivo.

β-gal transgene expression in vitro was determined after briefly fixing cells with 0.5% glutaraldehyde for 10 minutes then incubating in X-gal (5-bromo-4-chloro-indolyl-β-D-galactopyranoside) at 37° C. for at least 4 hours. β-galactosidase cleaves this substrate into an indigo compound, such that cells producing the β-galactosidase gene product are stained blue. β-gal expression was quantified by focusing on a representative area of the culture well under high power and recording the percentage of blue cells. In vivo, after the tumor was harvested and weighed, sections were incubated in the X-gal solution containing the detergents sodium deoxycholate (0.01%) and NP40 (0.02%). Representative tissue sections were then scored for percentage of blue-stained cells. Tumors infected with the parental ALVAC (not recombinant for the lacZ gene) were used as negative controls.

The luciferase assay from cell lysates was performed using a commercial luciferase assay kit (Promega Corp., Madison, Wis.) employing the manufacturer's recommendations. The Monolight 2010 Luminometer (Analytical Luminescence Laboratory, Ann Arbor, Mich.) was employed for the luciferase assay. Internal controls were performed by reading background luminescence for each assay as well as the periodic evaluation of the variation between replicates. Luciferase assays of infected subcutaneous tumors were performed after homogenizing each tumor with the Tissue Tearor (Biospeck Products, Inc.) in 0.5 mL of cell lysis buffer.

Tumor Inhibition Studies

RM-1 cells were injected subcutaneously in the backs of mice in a concentration of 5×10⁵ as described above. Approximately 10 days after tumor implantation a total of 8.4×10⁶ pfu of recombinant ALVAC vector was injected intratumorally. The ALVAC vectors used were the IL-2, IL-12, and TNFα constructs in equal concentrations (2.8×10⁶ pfu each) using an 18-gauge needle. The vectors absorbed by the gelatin sponge matrix were compared to three separate injections of the fluid-phase products (8.4×10⁶ total pfu) of 33 μL each. Other controls included parental ALVAC absorbed by the gelatin sponge matrix, matrix only, as well as a no treatment group.

RM-1 tumor outgrowth, determined by tumor size as a function of time, was measured approximately three times a week. Survival of the tumor bearing mice was also determined. Mice were sacrificed for humane reasons if a single tumor was greater than 25 mm in any dimension or if the mice appeared ill from the tumor burden. These experiments involved five mice per group and have been reproduced in four independent experiments.

Statistical Evaluation

Differences were analyzed by the rank sum test (Wilcoxon or Mann-Whitney) for non-parametric data such as differences in reporter assays or tumor volumes at individual time points. For the tumor outgrowth studies, a one-way ANOVA of log-transformed data was also used to compare groups. Survival data was compared utilizing the Kaplan-Meier method and analyzed for significance using the Cox Proportional Hazards Regression model. A computer software program was employed for all statistical analysis and all reportedp-values are two-sided. Significance was determined as ap-value of less than 0.05.

### Determination of Virion Concentration

The calculated volume delivered by the individual matrices was determined by the wet weight after absorption of the virus in the 18-gauge delivery needle. The mean wet and dry weights of at least five samples were assessed for each delivery matrix, and the results of three separate determinations are in Table 1.

TABLE 1
Calculation of volume delivered by individual matrices.

| | Chromic | Polyglycolic | Catgut | Gelatin |
|---|---|---|---|---|
| | catgut | acid | spacer | sponge |
| Wet weight | 2.15 ± 1.14 | 2.74 ± 0.98 | 4.47 ± 0.68 | 3.57 ± 0.78 |
| (mg) | | | | |
| Dry weight | 0.69 ± 0.01 | 0.56 ± 0.02 | 2.67 ± 0.05 | 0.93 ± 0.03 |
| (mg) | | | | |
| Volume | 1.46 ± 1.14 | 2.18 ± 0.97 | 1.80 ± 0.66 | 2.63 ± 0.80 |
| delivered | | | | |
| (μL) | | | | |

Note: weights are calculated as means±standard deviation of 3 separate experiments. Individual experiments consist of measurements of 5-10 matrices. Volume delivered is calculated from wet minus dry weight.

The known volumes absorbed by the individual matrices allowed comparisons to fluid-phase delivery both in vitro and in vivo by calculating the number of viral particles or plaque forming units. Thus, pfu delivery via either matrix or fluid was equivalent given the absorbable capacity of each matrix. Equivalency was verified in the gelatin sponge matrix system as determined by the particle count red at OD260.

### Delivery of Reporter Genes In Vitro

To examine the ability of each matrix to deliver the calculated virion concentration in cell culture, β-gal and the firefly luciferase expression of cells was determined 48 hours after infection with ALVAC-lacZ or ALVAC-luciferase. The virus delivered by the matrices was compared to the addition of a known MOI (pfu) of fluid-phase virus in cell culture.

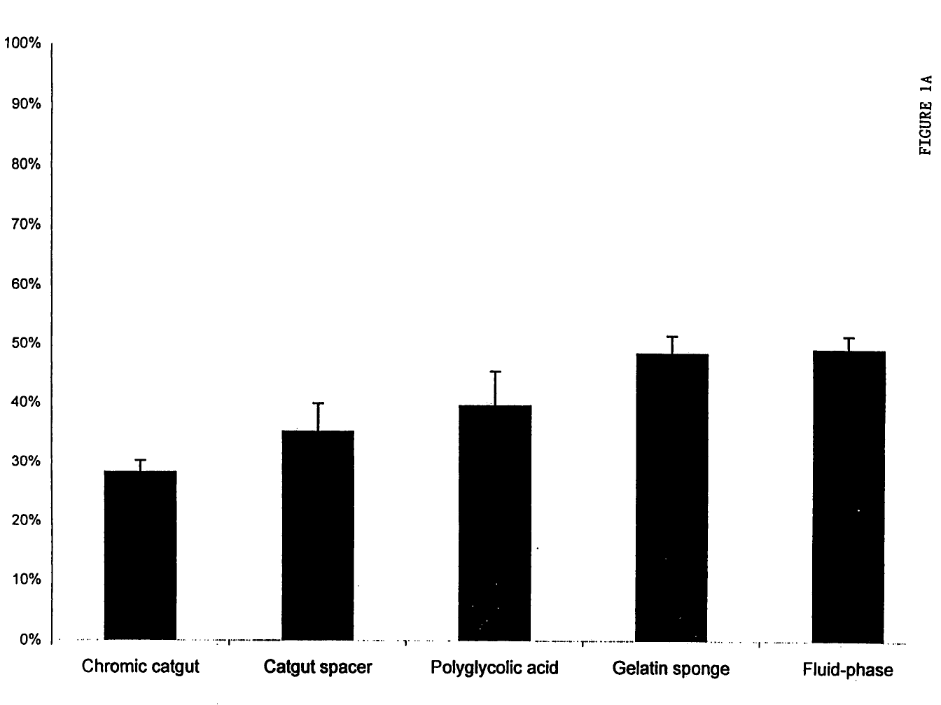

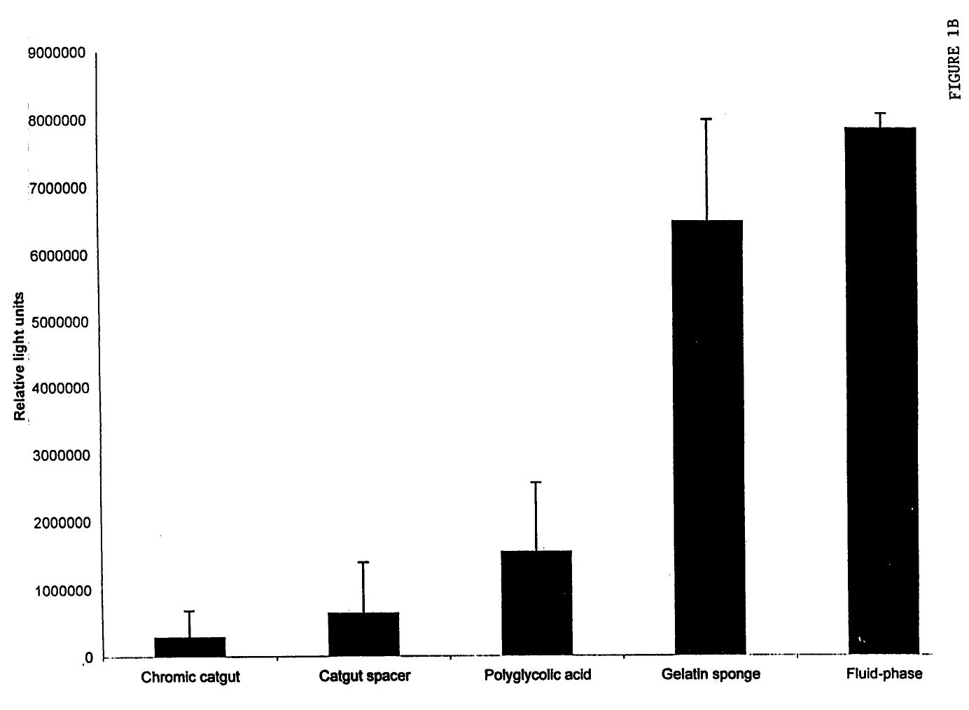

As shown in FIG. 1A, no significant differences were found with the percentage of cells expressing β-gal in the gelatin sponge matrix delivered groups versus the direct infusion groups. Interestingly, there was a trend to less β-gal expression for the other delivery systems, especially for the chromic catgut. These results were consistent over a wide range of MOI (10-200:1) for both tumor cell lines, RM-1 and MB-49.

Similarly, there was no significant difference between the relative luciferase activity of tumor cell lysates infected with ALVAC-luciferase delivered by the gelatin sponge compared to direct infusion (FIG. 1B).

### Transgene Expression in a Heterotopic Tumor Model

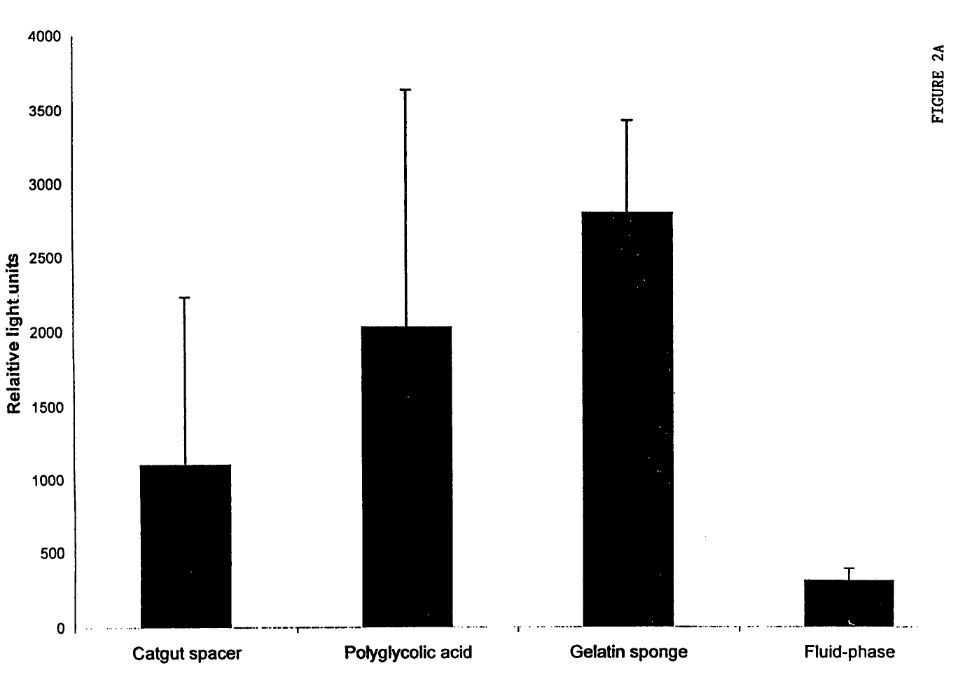

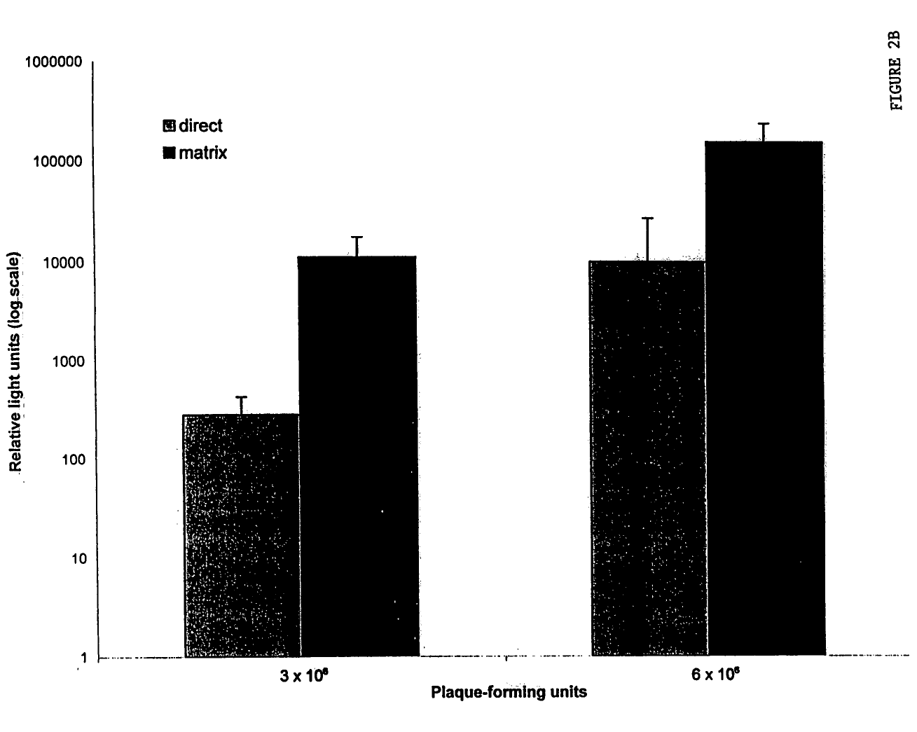

In order to determine the ability of these carrier systems to deliver the viral vectors in vivo, ALVAC-luciferase was first injected into established subcutaneous MB-49, either in fluid-phase or via the various delivery systems. The chromic system was not tested given its consistently poor transfer of virus into culture. Forty-eight hours after infection the tumors were harvested, and the luciferase assay was performed. The gelatin sponge matrix delivery resulted in significantly (p=0.03) enhanced gene expression over injection of the fluid-phase, while the other delivery systems demonstrated no statistically significant improvements (FIG. 2A).

Similar experiments in the heterotrophic RM-1 tumor model confirmed the ability of the gelatin sponge matrix to significantly improve the delivery of viral vectors compared to direct injection of the fluid-phase product. FIG. 2B represents this improved luciferase expression in the RM-1 tumor model at different doses (pfu) of the ALVAC-luciferase vector (note log scale for light units in FIG. 2B).

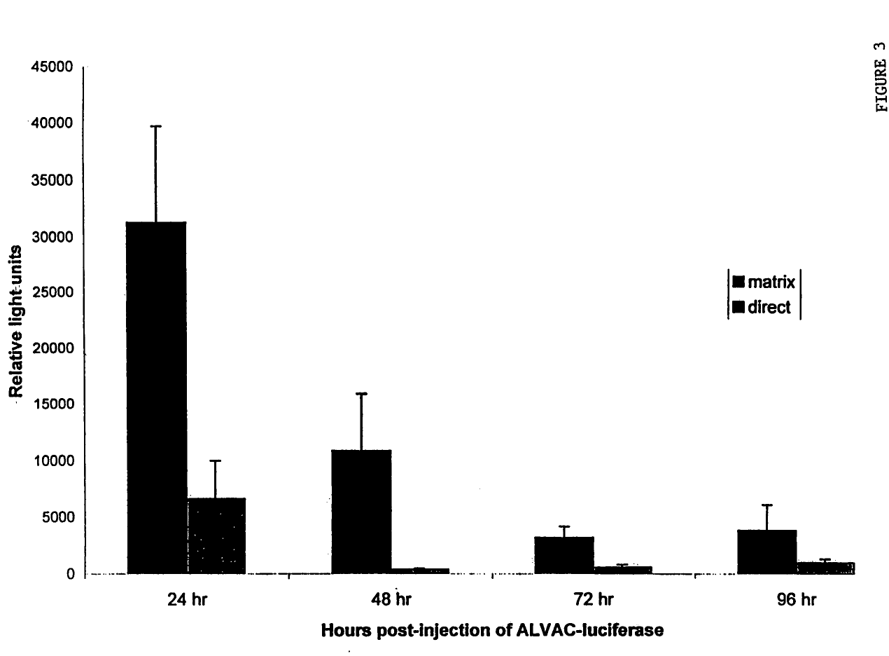

To determine if the improved in vivo gene expression consistently observed at 48 hours was conserved over time, subcutaneous RM-1 tumors infected with 3×10⁶ ALVAC-luciferase were harvested for luciferase assays at 24, 48, 72, and 96 hours after injection. Those tumors that were injected via the gelatin sponge matrix had remarkably greater luciferase activity than intra-tumoral fluid-phase injection at each period of time (FIG. 3).

The difference in each group was statistically significant, except at 96 hours, which approached significance given ap value of 0.058 (Wilcox rank sum test, two-tailed).

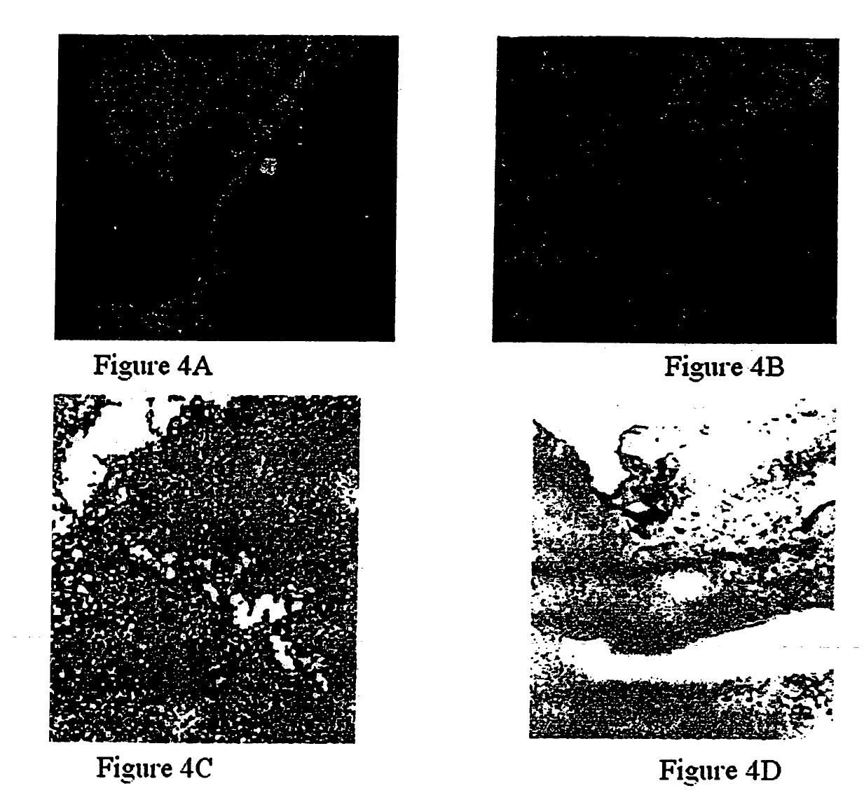

Enhanced gene expression was consistently demonstrated under various conditions (virion concentration, time) when the gelatin sponge matrix was employed to deliver the vectors. In order to determine if improved biodistribution throughout the tumor was partly responsible for this improved expression, heterotopic tumors were infected by ALVAC encoding reporter genes β-galactosidase or GFP. Viewing tumors under the fluorescent microscope after ALVAC-GFP infection revealed much brighter and more widespread fluorescence when the vectors were delivered by the gelatin sponge matrix (FIGS. 4A, 4B). For these comparisons, controls for background fluorescence were determined by infecting tumors with parental ALVAC. Similarly, fluid-phase injection of tumors by ALVAC-lacZ consistently revealed β-galactosidase activity only within a relatively narrow distribution along the needle tract. Delivery by the gelatin sponge matrix, however, resulted in substantially more widespread distribution (FIGS. 4C, 4D). These results were also controlled for endogenous β-galactosidase activity by staining tumors infected with parental ALVAC.

### RM-1 Tumor Outgrowth Studies

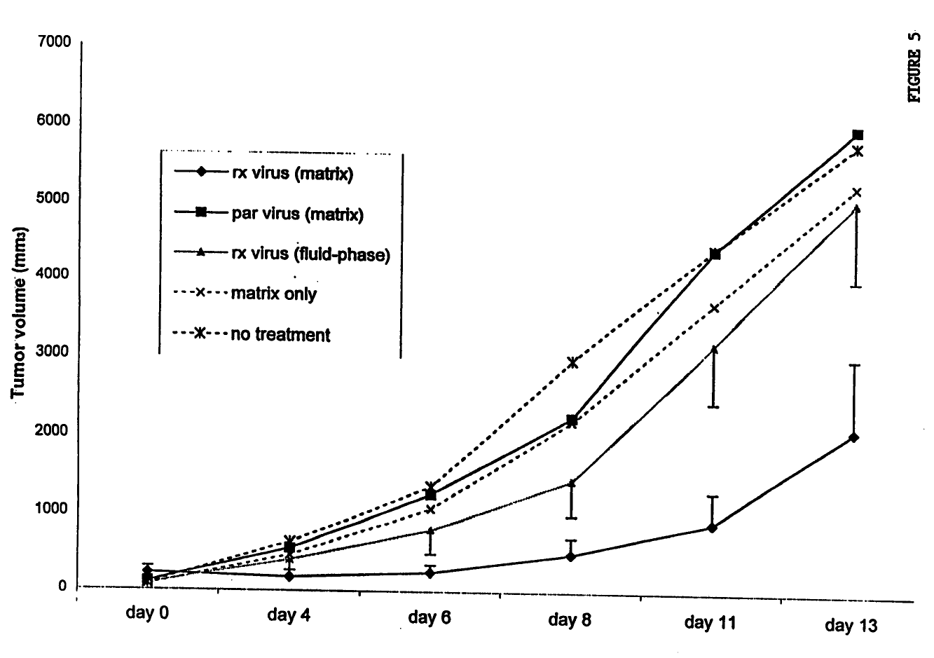

In order to determine if the enhanced gene expression found with the matrix delivery of vectors translated into improved biologic effect, established subcutaneous RM-1 tumors (mean volume 110 mm³) were treated with ALVAC IL-2, IL-12, and TNFα delivered in the gelatin sponge matrix or via fluid-phase injection. Control groups included parental ALVAC delivered via the matrix, a matrix only, and a group with no treatment. Significant tumor inhibition, as determined by tumor volume over time, was seen in the treatment group only when delivered by the matrix (FIG. 5).

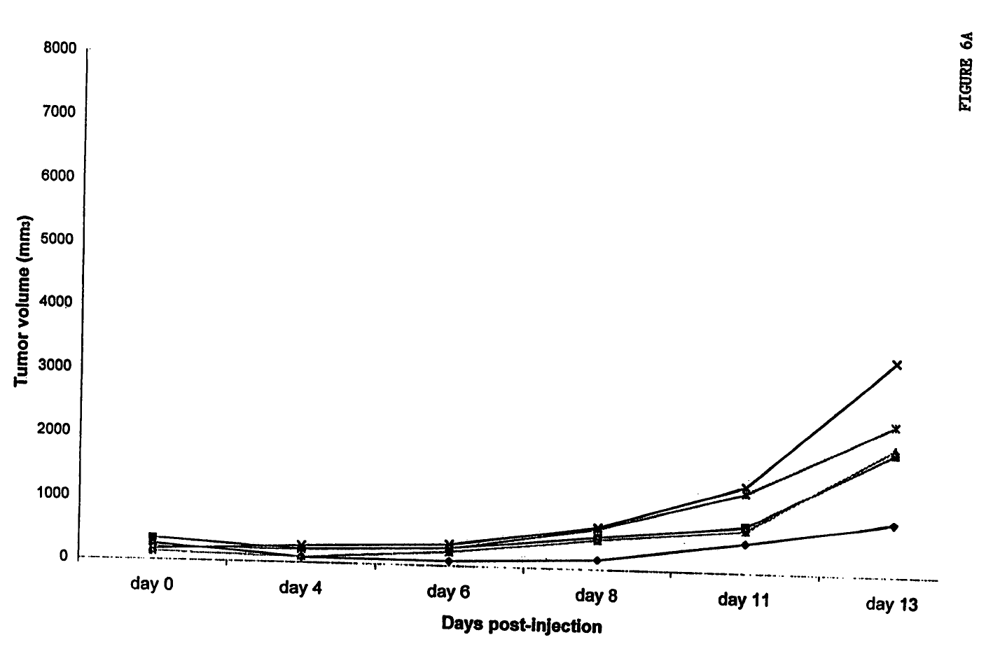

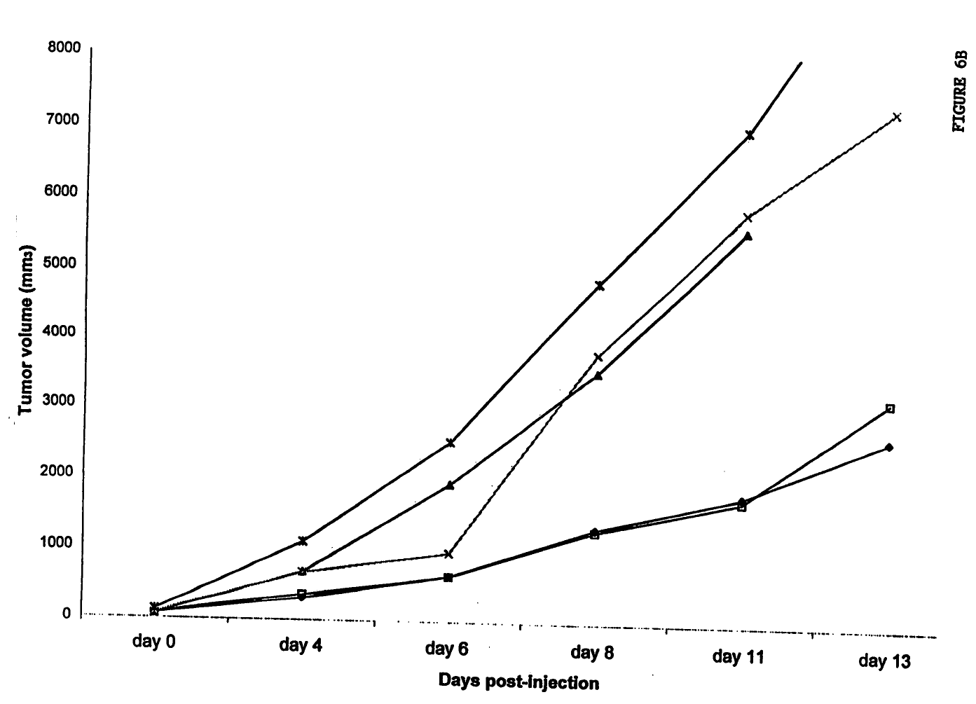

This tumor suppression was greatest within the first 6-7 days after infection with the recombinant virus, although the inhibitory effects remained significant through 13 days. Further comparison between groups was not possible because many control mice were euthanized after day 13. FIG. 6 represents the tumor outgrowth of the individual mice from the time of injection of recombinant virus.

Several tumors (3/5) in the treatment group delivered by the gelatin matrix (FIG. 6A) demonstrated regression in tumor size, although all tumors did eventually continue to grow.

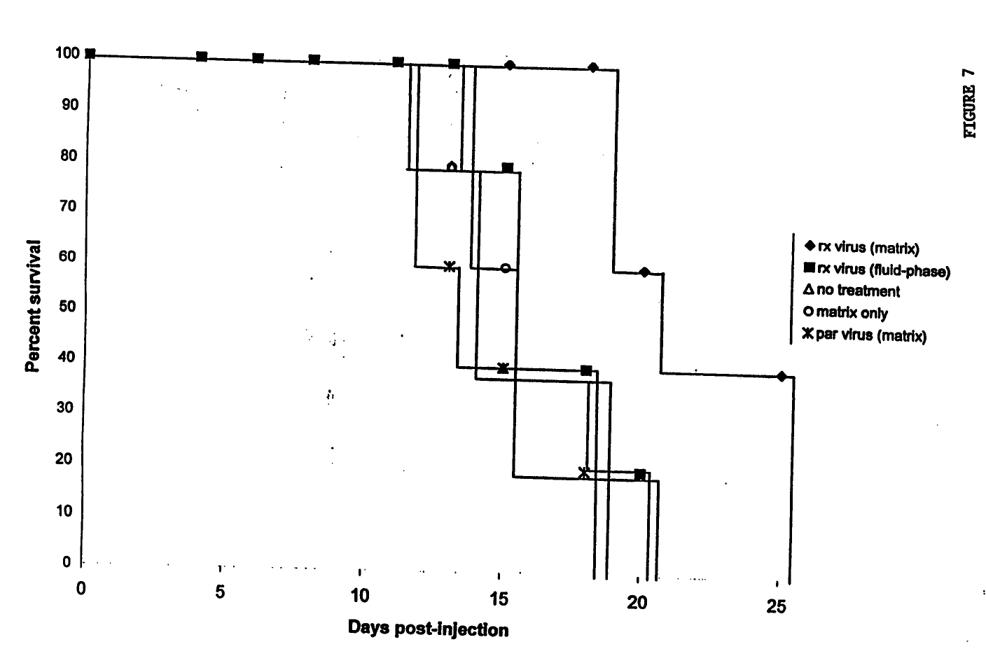

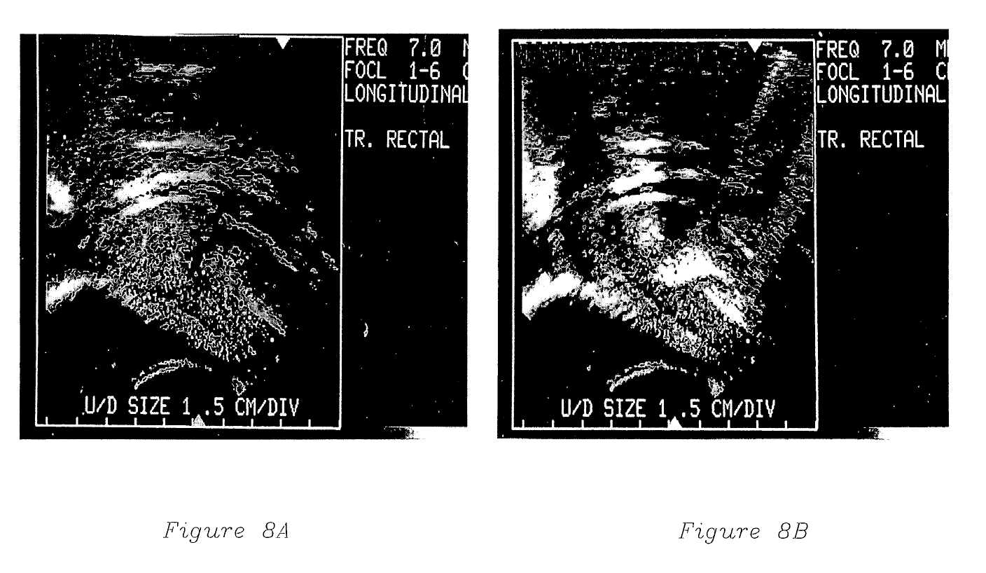

The regression observed in the gelatin matrix group contrasted to the effects of the fluid-phase delivery where all tumors continued to grow. Tumor volume at days 4 to 13 in the gelatin sponge matrix delivered group were statistically different compared to the fluid-phase injection (p<0.001), as well as the control groups (p<0.001). Similarly, a statistically significant difference was seen in the survival of those mice treated with the recombinant virus delivered by gelatin matrix as compared to fluid-phase injection (p<0.005; FIG. 7).

The differences observed between matrix and fluid-phase delivery have been confirmed in four separate tumor outgrowth experiments.

### Example 2

### Biomarker Distribution After Injection in the Canine Prostate

Materials and Methods

Animals. Adult male beagle dogs weighing approximately 15 kg. were used for all studies. The animals were allowed free access to food and water until the morning of the procedure and received one intramuscular injection of gentamicin pre-operatively. All animal studies were approved by the University of Iowa Animal Review Board and were performed in accordance with institutional guidelines.

Biomarkers/Gene Transfer Vectors. Carbon black as a particulate solution was used at full strength for these studies. ALVAC is a canarypox virus that can infect mammalian cells but is restricted to avian species for replication⁷ and has been shown to be a safe and effective vector in both humans and animals.⁸ The viral strain from which ALVAC was obtained was isolated from a pox lesion on an infected canary. The ALVAC vectors used in this study, including the reporter gene construct β-galactosidase (ALVAC-lacZ vCP326) and a B7 construct (ALVAC-B7, vCP268 {used as control for the β-gal staining}), were developed at Virogenetics Corporation (Troy, N.Y.).

Gelatin Sponge Matrix. Gelfoam (Pharmacia and Upjohn, Kalalmazoo, Mich.) is an absorbable gelatin sponge prepared from purified pork skin gelatin granules and is used as a hemostatic agent. The carrier was prepared in 6 mm lengths and subsequently injected through an 18-guage B-D spinal needle (Becton Dickinson and Co., Franklin, N.J.). As previously described for the carrier delivery for insulin, the virion concentration to be delivered by an individual matrix carrier was determined by weighing the matrices before and after viral absorption.⁹ To ensure that the gelatin sponge matrix was reliably delivering this weight-calculated number of viral particles, comparisons were made utilizing spectrophotometer particle determination by the optical density (OD) at 260 nm after digestion of the gelatin matrix. We have shown previously that the matrix does deliver the weight calculated virion concentration, based on particle determination, as well as in vitro infection using reporter gene constructs.

Delivery of viral vectors was also performed using Gelfoam powder (Pharmacia and Upjohn), a fine, light powder prepared by milling absorbable gelatin sponge. For these experiments, a known virion concentration was mixed in a ratio of 28 g of powder to 1 ml of virus in solution. The resulting viscous product was then injected into the prostate with an 18-gauge spinal needle. Previous experiments have shown improved delivery of viral vectors when delivered with the absorbable gelatin powder, equivalent to the gelatin sponge matrix (unpublished data).

Intraprostatic Delivery. After induction of anathesia by the intravenous injection of sodium pentobarbital, the dogs were placed in the supine position and the prostate was visualized with transrectal ultrasound (Bruel and Kjear) at 7.5 MHz. The first series of experiments were performed with carbon black administration in a total of 5 animals. Full strength carbon black was first infused into the canine prostate in a retrograde fashion utilizing a transurethral dual-balloon catheter. After assuring correct placement of catheter with transrectal ultrasound, carbon black was infused under pressure (100 cmH₂O).

We next evaluated the distribution of transrectal injection of carbon black in the prostate over time. Transrectal injection was performed in three dogs using a separate injection of 0.5 ml carbon black into both the right and left lobe using a 20-gauge spinal needle. The prostate of each animal was then harvested at different time points (1, 8 and 24 hours). In order to optimize the distribution of carbon black throughout the prostate, multiple transperineal injections were performed utilizing a method similar to that of brachytherapy protocols for prostate cancer. After transrectal ultrasound imaging of the prostate, a 20-gauge spinal needle was placed with the aid of a perineal grid secured to the rectal probe. Then with a Mick applicator, multiple injections of 0.1 ml carbon black were performed at 0.5 cm step intervals. The number of injections per pass was dictated by the transrectal ultrasound reconstruction of the prostate. All procedures were monitored by transrectal ultrasound and intraprostatic injection was confirmed by the appearance of a hyperechoic lesion (FIG. 8).

The second set of experiments were designed to evaluate the distribution of intraprostatic injections of a recombinant viral vector and compare the results to the distribution of carbon black. ALVAC-lacZ and ALVAC-B7 (recombinant for the B7 molecule and used as a negative control for β-gal expression) were injected in the fluid-phase (virus diluted in PBS) at a concentration of 1×10⁷ pfu/ml. Five injections of 1 ml each (for a total of 5×10⁷ pfu) were performed transrectally with a 20-gauge spinal needle. To evaluate any improved distribution when the viral vectors were delivered complexed in a solid-state, 1×10⁷ pfu of the ALVAC-lacZ or ALVAC-B7 was absorbed by a gelatin sponge matrix and delivered into the prostate transrectally. Five separate implants per prostate were performed for a total virion concentration of 5×10⁷. Similarly, 5 separate injection of 1×10⁷ ALVAC-lacZ delivered in the Gelfoam powder (0.2 ml volumes) were performed. A single animal was used for each experimental group.

Histopathology and Reporter Gene Assays. After intraprostatic administration of carbon black, the dogs were euthanized at various time points (described for individual experiments). At this time, the bladder and prostate was harvested and a pelvic lymphadenectomy performed. The prostate and lymph nodes were immediately placed in a formalin solution and paraffin embedded. A wide sampling of transverse sections, perpendicular to the urethral axis, were routinely processed and stained with hematoxylin and eosin.

The procedure for those prostates injected with the ALVAC viral vectors differed slightly. All prostates were harvested after 24 hrs. and fixed with 0.05% glutaraldehyde. After rinsing with PBS, the sectioned samples of prostate were then incubated with X-gal solution (5-bromo-4-chloro-indolyl-β-D-galactopyranoside) at 37° C. for 4 hours. β-galactosidase cleaves this substrate into an indigo compound, such that cells producing the β-galactosidase gene product are stained blue. Previous studies revealed that re-staining the frozen sections with the X-gal solution overnight optimized the level of blue staining of cells. All tissues were stained in the same fashion in order to accurately compare gene expression. Representative frozen sections were then counterstained with nuclear fast red and evaluated for percentage of blue-stained cells. Prostates infected with ALVAC-B7 (injected in the fluid-phase, as well as gelatin sponge matrix delivered) were used as negative controls.

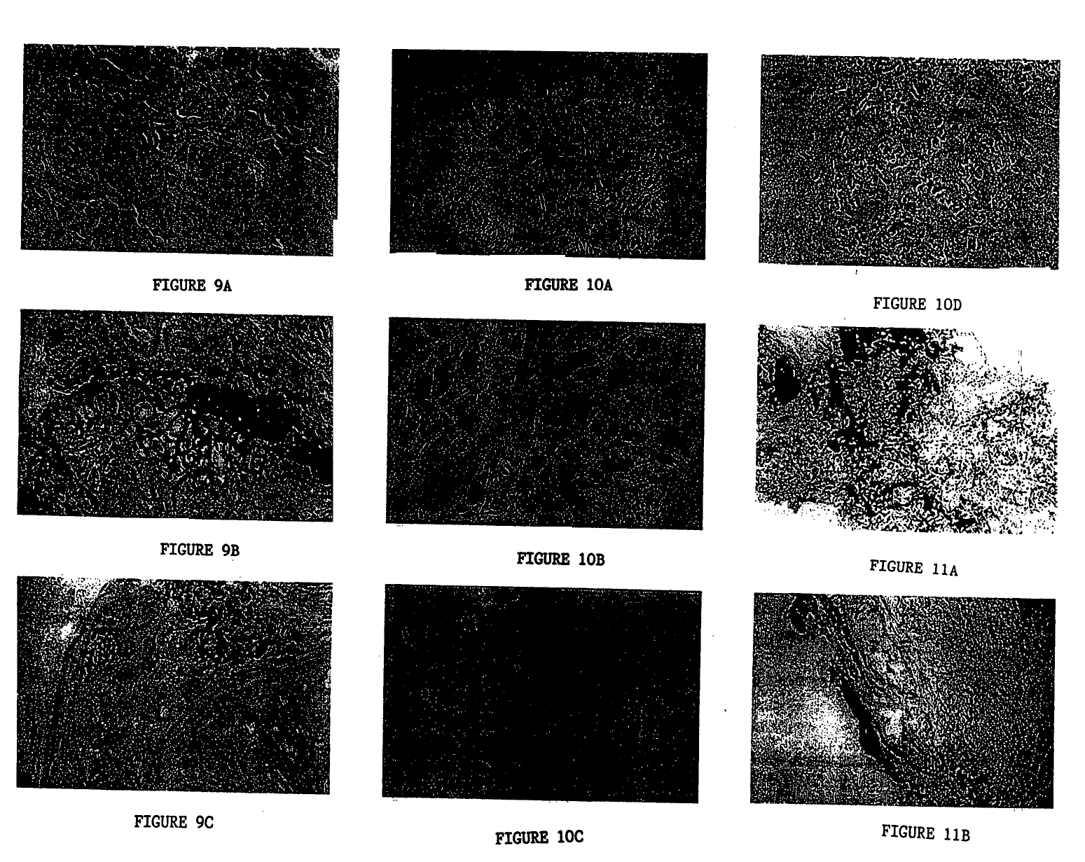

Transurethral Delivery of Carbon Black. As depicted in FIG. 9A carbon black was detected in several acini, as well as some large ducts, mostly in the periphery of the prostate when delivered transurethrally. However, multiple sections throughout the prostate revealed poor distribution of carbon black, with only a minority of ducts/acini involved. Although the carbon black was able to reach a limited number of acini in the periphery of the gland, there was no evidence of further periacinar spread 24 hours after injection.

Results

Transrectal/Transperineal Intraprostatic Injection of Carbon Black. Single injections of 0.5 ml carbon black into either lobe of the prostate resulted in localized collections of carbon black in the stroma, as well as what was determined to be the needle tract. Radiating from these collections, a minor amount of carbon black could be detected tracking in periacinar tissue planes, apparently in the direction of the prostatic capsule (FIG. 9B). This periacinar tracking at 24 hours after injection was not appreciably greater than after 1 hour and was limited in distribution to the site of injection. In each of the transrectal injections, carbon black could be visualized tracking out of the prostatic capsule along the puncture path.

In an attempt to improve on the observed localized distribution of a single transrectal injection of biomarker, multiple injections of 0.1 ml carbon black were performed transperineally. These injections were evenly spaced (0.5 cm steps) utilizing prostate brachytherapy methodology under transrectal ultrasound guidance. Because of the mobility of the canine prostate, two 20-gauge spinal needles, placed into the prostate, were used for stabilization during injection. Not all of the planned passes were possible because of the location of the prostate relative to the pubic arch. The prostate was harvested 24 hrs. after injection and it was noted that a large quantity of the injected carbon black was localized in the periprostatic tissue, and the prostate itself was encompassed by a thick layer of dye contained by a pseudo-capsule. In fact, a minimal amount of carbon black was observed within the prostate itself on histopathology, with the majority located outside of the prostatic capsule (FIG. 9C). No carbon black was visualized in the pelvic lymph nodes in any of the experimental conditions, up to 24 hours after intraprostatic injection.

Transrectal Injection of Viral Vector. No β-gal expression was detected after injection of the ALVAC-B7 construct, either in the fluid-phase or complexed to the gelatin sponge matrix (FIGS. 10A and 10B). The tissue was sectioned numerous times to ensure extensive sampling of the entire prostate. The frozen sections were stained twice in the X-gal solution to ensure detection of any endogenous β-gal activity.

After injection of the ALVAC-lacZ construct in the fluid-phase, areas of β-gal gene expression were easily identified after staining. These blue-stained cells appeared to be mostly located in the stroma, although occasional epithelial cell staining was detected in acini (FIG. 10C). The distribution of reporter gene expression was very localized to what appeared to be the site of injection without much spread at 24 hours after injection. Similar to the results of the carbon black injections, β-gal staining could be detected in what appeared to be the puncture path with extravasation out to the prostatic capsule.

In contrast, significantly greater β-gal expression was observed when the ALVAC-lacZ vector was delivered complexed to the gelatin sponge matrix (FIG. 10D). Defined regions of gene expression could be detected throughout the prostate, although with much greater area of distribution compared to the fluid-phase delivery. As observed with the fluid-phase injection, β-gal expression could be seen in cells located in the stroma; however, much greater expression was observed in epithelial cells of the acini. Similarly enhanced gene expression was observed when the ALVAC vector was delivered with the absorbable gelatin powder compared to the fluid-phase injection (FIG. 11A) although more β-galactosidase expression was demonstrated along the prostatic capsule than the gelatin sponge matrix (FIG. 11B).

Similar to the carbon black results, no 1-gal expression was detected in any of the pelvic lymph nodes within 24 hours of injection.

We have previously shown that this gelatin matrix mediated enhanced gene expression when compared to the fluid-phase injection of the viral vector in pre-established subcutaneous tumor nodules. As well, this enhanced gene transfer was shown to translate into improved biologic effect in the murine prostate cancer model using a cytokine-based immunotherapy protocol.

In this experiment, the delivery of the ALVAC-lacZ vector in the canine prostate was markedly improved when associated with the gelatin sponge carrier. Although still regionalized to the site of placement, gene expression was qualitatively higher with a much wider area of distribution as compared to the fluid-phase injection. This was accomplished with injecting the same viral concentration. Enhanced delivery was also demonstrated using the non-cross-linked Gelfoam powder as the delivery agent.

### Example 3

Adenovirus/PSA Immunization Combined with ALVAC Cytokine Gene Delivery Induces Destruction of Established Prostate Tumors

In humans, synthetic PSA peptides containing consensus HLA-A2-binding motifs have been shown to induce PSA-specific CD8+ cytotoxic T lymphocytes (CTL) following in vitro incubation with peripheral blood lymphocytes from normal HLA-A2 donors. (Correale P, et al, “In vitro generation of human cytotoxic T lymphocytes specific for peptides derived from prostate-specific antigen”, J Natl Cancer Inst 1997; 89:293-300.) The peptide-reactive CTL lysed PSA-positive, HLA-A2 positive human prostate carcinoma cell (LNCaP) targets or peptide-absorbed HLA-A2 cells.

The data reported by these laboratories suggest that PSA may be a useful antigen for the immunotherapy of prostate cancer. However, the demonstration of protective immunity or the ability to destroy established tumor in relevant prostate cancer models is lacking. In other model systems, immunotherapy has been shown to have benefit in generating protective responses against tumor challenge, and in selected models, to control established tumors. Both protection and regression of established tumors were largely due to the presence of antigen specific CD8+ cytotoxic T lymphocytes (CTL) that efficiently eradicate primary and metastatic tumor cells. Thus, we have developed Ad/PSA as an immunizing vector to investigate the potential of PSA-specific immunity to mediate antitumor activity in a TGFβ-secreting prostate cancer model, RM11. Using this model, we have shown that immunization of BALB/c mice with Ad/PSA, but not Ad/lacZ, generates a significant humoral and cellular anti-PSA response that protects the mice from challenge with RM1/PSA, but not RM11/neo. Through depletion studies, we have identified the primary effector cell as a CD8+ T cell. Yet, in spite of the robust protective anti-PSA responses induced by Ad/PSA immunization, 3 day established tumors were poorly controlled by Ad/PSA immunization alone. However, we found that large established tumors were readily eliminated in 73% of mice primed with Ad/PSA followed in 7 days by an intra-tumoral injection of recombinant ALVAC encoding IL-12, IL-2, and TNF-α.

Methods and Materials

Non-lytic adenovirus expressing human PSA. The PSA cDNA provided by Donald Tindall, Mayo Clinic, Rochester, Minn., was placed 3′ to the CMV promoter in a shuttle vector containing Ad5 DNA. The sequence inserted was the pre-pro form of PSA described by Lundwall (Lundwall A, “Characterization of the gene for prostate-specific antigen, a human glandular kallikrein”, Biochem Biophys Res Comm 1989; 161:1151-1159) that encodes 262 amino acids with a predicted molecular weight of 28.8 kDa. Using methods previously described (Davidson B L, “Expression of Escherichia coli beta-galactosidase and rat HPRT in the CNS of Macaca mulatta following adenoviral mediated gene transfer”, Exp Neurol 1994; 125:258-267.), the shuttle vector and E1a-E1b deletion mutant Ad5 DNA were transfected into HEK 293 cells, and recombination between the DNA species was allowed to occur. The amplification and purification of Ad/PSA was performed by the University of Iowa Gene Transfer Vector Core as previously described. (Graham F L, “Characteristics of a human cell line transformed by DNA from human adenovirus type 5” J Gen Virol 1977; 36:59-74.) Ad/lacZ used as a control was also obtained from the Gene Transfer Vector Core and is previously described. (Jaffe H A, et al, “Adenovirus-mediated in vivo gene transfer and expression in normal rat liver” Nature Genet 1992; 1:372-378.)

RM11/PSA cell line. The myc- and ras-transformed BALB/c (H-2^{d}) RM 11 prostate cell line was obtained from Timothy Thompson, Baylor College of Medicine. (Thompson T C, “Genetic predisposition and mesenchymal-epithelial interactions in ras+myc-induced carcinogenesis in reconstituted mouse prostate” Mol Carcinog 1993; 7:165-179.) The PSA cDNA was subcloned into pHβApr-1-neo. (Gunning P, “A human β-actin expression vector system directs high-level accumulation of antisense transcripts”, Proc Natl Acad Sci USA 1987; 84:4831-4835) RM11 cells were electroporated with this plasmid in a Gene Pulser (BIO-RAD, Hercules, Calif.) at 300 volts, 960 μF, τ=18.0 ms in a 0.4 cm gap electrode. After 24 hr. growth, 400 μg/ml of active G418 (Calbiochem, La Jolla, Calif.) was added to the medium. Fourteen days later, neo^{r} cells were cloned by limiting dilution. Clones E5 and E6 were chosen for in vitro and in vivo studies. Both cloned lines function as in vitro targets for CTL, but only RM11/PSA clone E6 grows at a rate comparable to control RM11 tumors in vivo, and as a result, is the only clone used for in vivo studies. PSA secretion by the clones was assessed by immunoassay. The cells are adherent, express MHC class I which can be upregulated with IFNγ, have a doubling time of approximately 12 hr., and express TGFβ (data not shown). PSA secretion by cell lines was verified by immunoassay (Table 1). To generate a control cell line for RM11/PSA, RM11 cells were electroporated and cloned using the same conditions as for RM11/PSA, except the DNA used for transfection was the pHβApr-1-neo vector without the PSA cDNA (RM11/neo). RM11/neo and RM11/PSA cell lines are grown in DMEM (GibcoBRL, Grand Island, N.Y.)+10% fetal bovine serum (HyClone, Logan, Utah), 100 units/ml penicillin, 100 μg/ml streptomycin, 0.5 mg/ml Gentamicin, 2 mM L-glutamine, 0.01M HEPES, and 1 mM sodium pyruvate (Sigma, St. Louis, Mo.).

P815/PSA cell line The DBA/2 (H-2d) mastocytoma cell line was electroporated with the same PSA plasmid and equipment as RM11 at 300 volts, 960 μF, τ=15.7 ms. After 24 hr. growth, 600 μg/ml active G418 was added to the medium. Fourteen days later, neo^{r} cells were cloned by limiting dilution. PSA secretion was assessed as with RM11/PSA clones (Table 1). P815/PSA cells are cultured in RPMI-1640 (GibcoBRL, Grand Island, N.Y.) and supplemented with identical components as DMEM described above. This cell line is used for restimulation of splenocytes for in vitro assays.

Viral immunization The viral particles were diluted in PBS and injected intraperitoneally (ip.) in 0.1 ml volumes into BALB/c mice at the indicated pfu/animal.

Measurement of PSA PSA in supernatant fluids was measured by immunoassay (IMₓ, Abbott Laboratories, North Chicago, Ill.).

Isolation and detection of anti-PSA T cells To detect and expand any PSA-specific CTL, spleens were harvested 14 days after virus injection, cell suspensions prepared, and splenocytes isolated from red blood cells by Fico/Lite-LM (Atlanta Biologicals, Norcross, Ga.) separation. Splenocytes were incubated in 24-well plates in Click's medium (Sigma, St. Louis, Mo.) at 1×10⁷ cells/well together with P815/PSA at 4×10⁵ cells/well as stimulators (25:1)+10 IU/ml rhIL-2. P815/PSA cells were treated with 50 μg/ml mitomycin C (Sigma) for 30 min. After 5 days, live splenocytes were harvested by Fico/Lite-LM separation and used as effectors in a 4 hr. Na⁵¹CrO₄ (Amersham Corporation, Arlington Heights, Ill.) release assay. Ten thousand targets were used per well. Supernatant fluids were harvested and measured on a gamma counter (Beckman Instruments, Inc., Palo Alto, Calif.). Percent lysis was calculated by the following formula:

$$\frac{sample cpm - spontaneous release cpm}{maximum release cpm - spontaneous release cpm} \times 100$$

For proliferative assays, splenocytes were incubated in 96-well plates at 5×10⁵ cells per well+/−10 μg/ml human PSA purified from semen (gift from Robert Vessella, University of Washington). After 4 days, cells were labeled for 6 hrs. with 1 μCi ³H-thymidine (Amersham Corporation, Arlington Heights, Ill.). Cells were then collected onto glass fiber filter paper using an automated cell harvester (Skatron Instruments, Norway) and counted in a β-scintillation counter (Beckman Instruments, Inc.).

Anti-PSA antibody detection Ninety-six-well plates, coated with streptavidin (Labsystems Oy, Helsinki, Finland), were blocked with 2% BSA+0.2% tween 20 in PBS. Plates were then washed 3× with PBS-tween 20 and biotinylated PSA (a generous gift from T. J. Wang, Beckman Coulter, Brea, Calif.) added for 1 hr. at room temperature (RT). Plates were washed 3× again and serum samples added at appropriate dilutions at RT. One hour later, plates were again washed 3× and HRP-labeled goat anti-mouse Fc (Sigma) was added per well. After 1 hr at RT, plates were washed 3× and the chromogen o-phenylenediamine (Sigma) was added to the wells. After 30 min. at RT, the sample reactions were stopped with 4 N H₂SO₄ and absorbances read at 490 nm. To determine the amount of anti-PSA antibody present in the serum, sample absorbance values were compared to a standard curve derived from known concentrations of mouse anti-human PSA (Dako Corporation, Carpinteria, Calif.).

In vivo depletion of CD4+ and CD8+ T cells For in vitro assays, 100 μg of 2.43 anti-CD8 antibody or GK1.5 anti-CD4 antibody were injected ip. into mice for three consecutive days before splenic harvest. For in vivo tumor studies, mice were injected ip. with 100 μg of 2.43 or GK1.5/mouse for three consecutive days before tumor was implanted sc. on the back and thereafter every other day for the duration for the experiment. The anti-NK antibody anti-asialo GM1 (asGM1; Wako Bioproducts, Richmond, Va.) was injected once at 25 μg/mouse three days before tumor challenge and thereafter every 4 days at 25 μg/mouse. The control antibody used was a non-specific rat IgG2b (SFR8-B6, American Type Culture Collection, Manassas, Va.). All injections volumes were 100 μl.

Verification of T cell and NK cell depletion Depletion of T cells was assessed on the day of tumor challenge by flow cytometric analysis of spleen cells stained with 2.43 or GK1.5 followed by FITC-labeled goat antibody to rat IgG. For each analysis, >99.5% depletion of the desired population was achieved. NK cell depletion was verified by ⁵¹Cr release assays against NK-sensitive YAC-1 targets. Mice treated with anti-asGM1 showed no significant lytic activity against YAC-1 cells (data not shown).

Injection of tumor cells RM11/PSA and RM11/neo, in log-phase growth, were removed by trypsin treatment, washed, and resuspended in serum-free DMEM. Mice were given sc. injections on the back in a volume of 100 μl.

Gene transfer vectors ALVAC is a canarypox virus that can infect mammalian cells but is restricted to avian species for replication (30) and has been shown to be a safe and effective vector in both humans and animals. ALVAC vectors encoding either murine interleukin (IL)-2 (vCP275), murine IL-12 (vCP1303) and human tumor necrosis factor (TNF-α) (vCP245), as well as the parental ALVAC (CCpp), were developed at Virogenetics (Troy, N.Y.).

Tumor destruction studies RM11/PSA cells, clone E6, were injected sc. in the flanks of mice in a concentration of 1×10⁵ as described above. Three days after tumor implantation mice were immunized ip. with a total of 1×10⁸ pfu of either Ad-PSA or Ad-lacZ. Seven days later (10 days after tumor implantation) palpable tumors from 500-1000 mm³ were injected with 1×10⁷ pfu of each recombinant ALVAC vector encoding IL-2, IL-12 or TNF-α (total ALVAC dose=3×10⁷ pfu). Controls for the ALVAC cytokine vectors included intra-tumoral injection of 3×10⁷ ALVAC parental virus. All vectors utilized for intra-tumoral injections were delivered in a volume of 0.15 ml and mixed with Gelfoam powder, which acted as a solid-state carrier system. Gelfoam (Pharmacia and Upjohn, Kalalmazoo, Mich.) is an absorbable gelatin sponge prepared from purified pork skin gelatin granules and is used as a hemostatic agent. Gelfoam powder is a fine, light powder prepared by milling absorbable gelatin sponge. For these experiments, a known virion concentration was mixed in a ratio of 30 mg of powder to 1 ml of virus in solution. The resulting viscous product was then injected into the palpable tumors. Tumor outgrowth, determined by tumor size as a function of time, was measured approximately three times a week. Survival of the tumor bearing mice was also determined. Mice were sacrificed for humane reasons if a single tumor was greater than 25 mm in any dimension or if the mice appeared ill from the tumor burden. All experiments were repeated twice and experimental groups consisted of 5-6 mice.

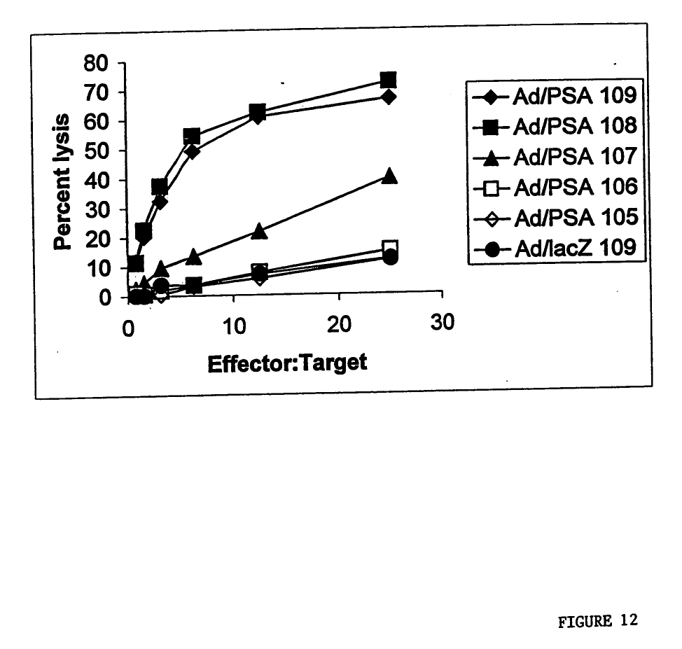

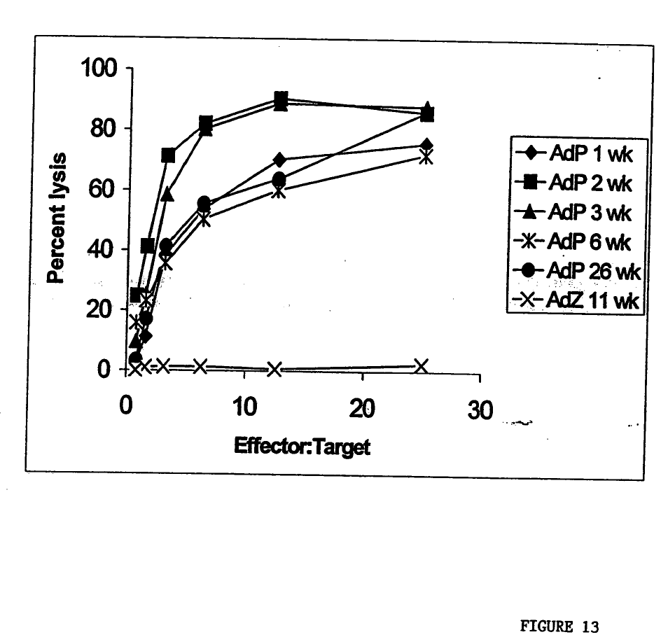

Generation and verification of deletion mutant recombinant adenovirus type 5 expressing human prostate specific antigen. The PSA cDNA was subcloned into an adenoviral shuttle vector (FIG. 12) which was used to generate infectious but replication-deficient E1a-E1b deletion mutant Ad/PSA particles as referenced in Materials and Methods. We assayed the ability of these purified particles to induce PSA production in the permissive HEK 293 cells and in the non-permissive RM 11 cell line by immunoassay. Ad/PSA induced significant PSA expression in both cell lines (FIG. 13).

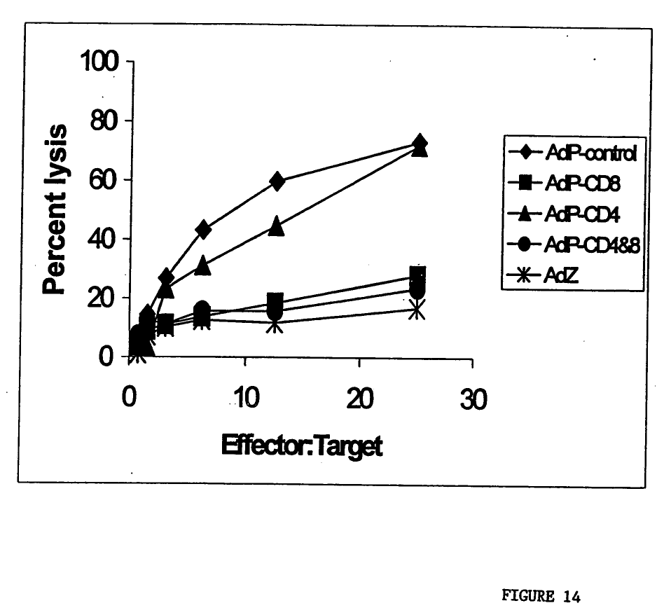

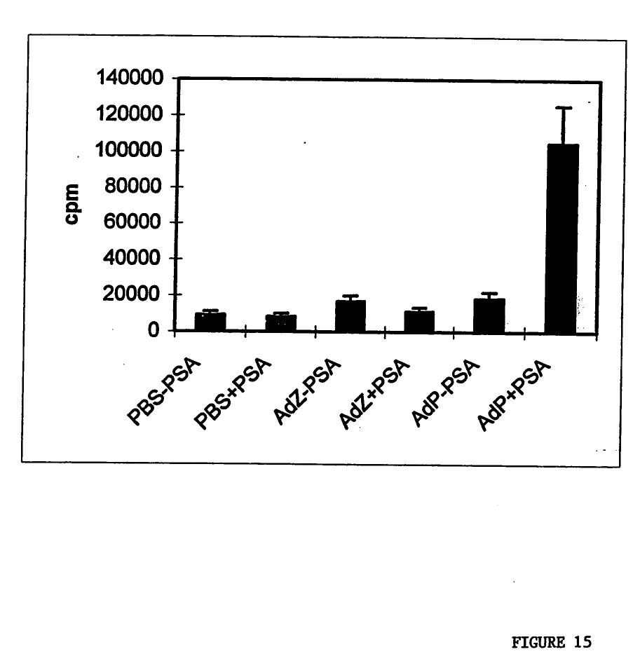

Characterization of the immunizing potential of Ad/PSA To determine if Ad/PSA could induce a CD8+ T cell response against PSA, mice were given ip. injections of 1×10⁹ plaque-forming-units (pfu) Ad/PSA or Ad/lacZ. Spleens were harvested 14 days later and cultures were established for CTL expansion as described in Materials and Methods. ⁵¹Chromium release assays show that splenocytes isolated from BALB/c mice immunized with Ad/PSA and cultured as described above can mount a PSA-specific lytic response against PSA-expressing targets, validating Ad/PSA as an antigen delivery vector suitable for immunization. To determine the optimum dose-dependent activation of PSA-specific cytolytic activity, Ad/PSA was serially diluted in log decrements to yield doses of 1×10⁹ to 1×10⁶ pfu per mouse. These doses were injected ip. and the spleens were harvested 14 days later. Cultures were established for CTL expansion as described in materials and methods. The results show that, as measured in vitro, 1×10⁸ to 1×10⁹ pfu induced maximal lytic activity, 1×10⁷ pfu induced a detectable CTL response, while lower doses were ineffective (FIG. 14). We also measured the duration of the anti-PSA response. Mice were immunized with 1×10⁹ pfu of Ad/PSA or Ad/lacZ 1 to 5 weeks prior to the assay date. All spleens from weeks 1 through 5 were then harvested at the same time to eliminate possible variations between assays. The 26 week data was a separate harvest. FIG. 15 shows that 2-3 weeks is the optimum time to detect anti-PSA cytolytic activity in vitro using bulk cultures, although activity remained significant as far out as 26 weeks.

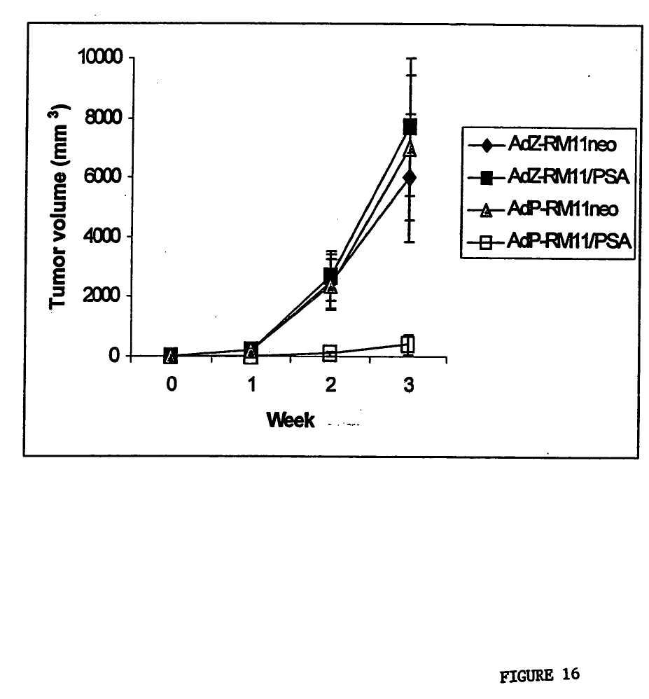

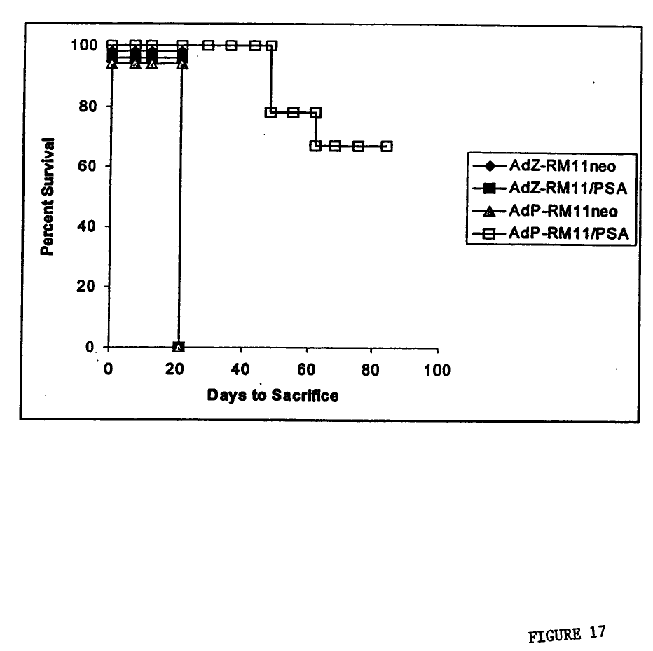

Evidence that the PSA-specific lysis is mediated by CD8+ CTL was obtained from experiments in which specific T cell populations were depleted or blocked. Splenocytes isolated from Ad/PSA-immunized mice depleted of CD8+ T cells in vivo by ip. injection of the monoclonal antibody 2.43 do not lyse PSA-expressing targets (FIG. 16). Furthermore, the addition of 2.43 antibody in lytic assays blocked the ability of splenocytes from normal Ad/PSA-immunized mice to lyse PSA-expressing targets (data not shown). In vivo depletion of CD4+ T cells with ip. injection of the monoclonal antibody GK1.5 did not abrogate this lysis. Depletion of T cells was verified by flow cytometry (FIG. 17).

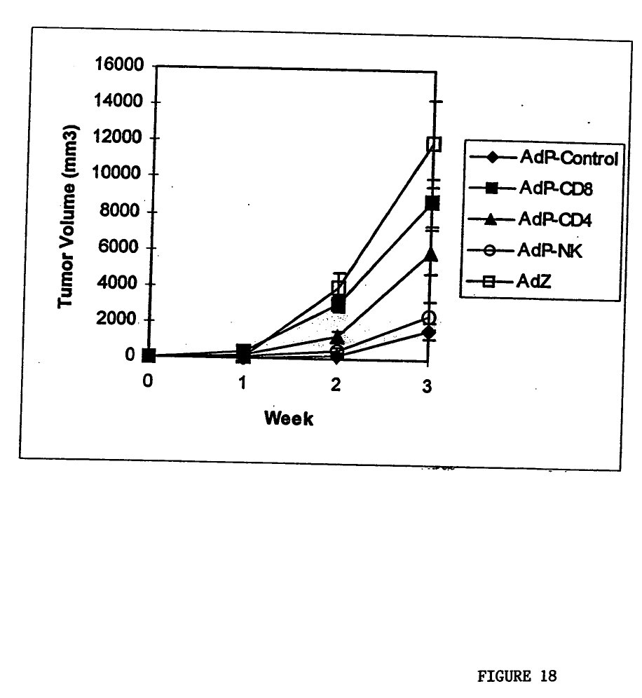

Further studies were performed to determine if PSA-specific lymphoproliferative and antibody responses were also generated by Ad/PSA immunization. Only splenocytes from Ad/PSA-immunized mice proliferated in response to PSA (FIG. 18). To detect the presence of anti-PSA antibodies, sera from Ad/PSA and Ad/lacZ-immunized mice were collected and assayed by enzyme-linked immunosorbent assay. Three different experiments showed that mice immunized with Ad/PSA but not Ad/lacZ produce high amounts of anti-PSA antibody.

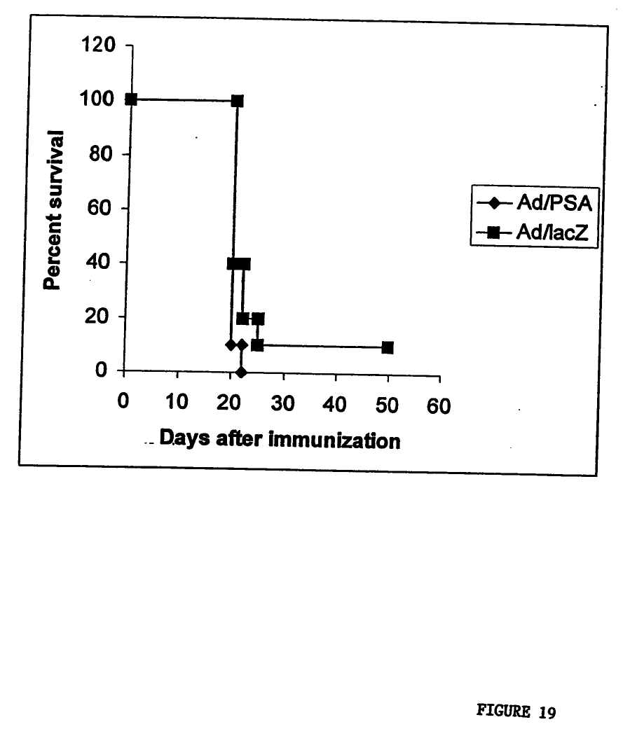

BALB/c mice immunized with Ad/PSA are protected from challenge with PSA-expressing prostate tumors To investigate whether the in vitro activity against PSA-expressing targets would correlate with in vivo protection, we immunized BALB/c mice ip. with 1×10⁹ pfu Ad/PSA or Ad/lacZ and 14 days later challenged them with a sc. injection of 1×10⁵ RM11/neo or RM11/PSA cells, clone E6. Tumor volumes were calculated weekly by multiplying measurements of height, length, and width. The data show antigen-specific, PSA-dependent, protection against tumor challenge (FIG. 19a). Inhibition of tumor growth was observed only when mice were immunized with Ad/PSA and challenged with RM11/PSA. These mice also showed significantly enhanced survival over controls. Sixty-seven percent of Ad/PSA-immunized RM11/PSA-challenged mice remained tumor-free at 80 days, whereas all of the control mice were euthanized at 21 days due to large tumor growth (FIG. 19b).

CD8+ T cells are largely responsible for mediating the anti-tumor effects Our data clearly show that antigen-specific immunization by Ad/PSA results in the development of protective immunity. In vitro characterization of immune responses induced by Ad/PSA showed antigen-specific lysis of PSA-expressing targets by CD8+ T cells, PSA-specific lymphoproliferation, which implicates reactive CD4+ T cells, and PSA-specific antibody production. To determine which effector arm is responsible for mediating the protective effects against PSA-expressing prostate tumor growth, mice were immunized ip. with 1×10⁹ pfu Ad/PSA or Ad/lacZ. On days 11, 12, and 13 after immunization, groups of 6 Ad/PSA-immunized mice were injected with the control antibody SFR8-B6, the CD8+ T cell-depleting antibody 2.43, or the CD4+ T cell-depleting antibody GK1.5. The NK-depleting antibody anti-asialo GM1 was injected on day 11 and every four days thereafter. At day 14, all mice were challenged sc. with 1×10⁵ RM11/PSA tumor cells. One animal from each group of mice was euthanized to confirm that the depleting antibodies did indeed deplete the cell populations they target.

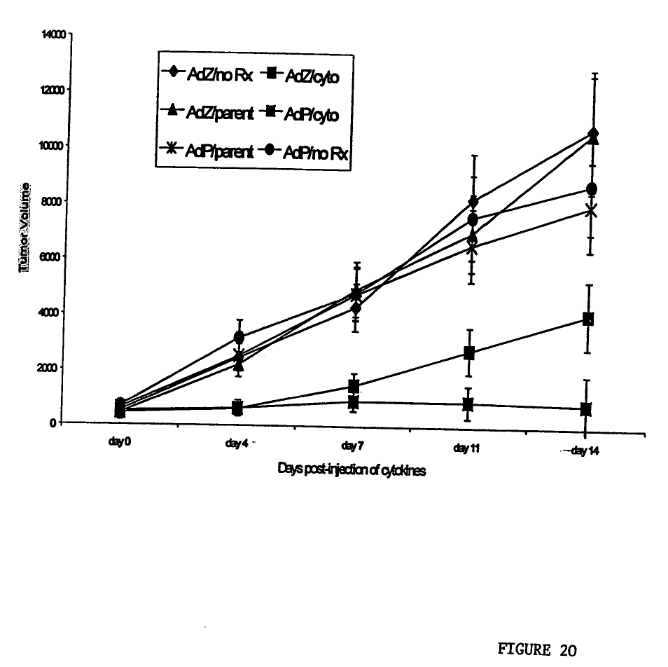

Flow cytometry shows that the CD4 and CD8-depleting antibodies achieved >99.5% depletion of T cells compared to the injected control rat IgG antibody SFR8-B6. Likewise, functional studies showed an absence of NK lytic activity as measured by the ability of anti-asialoGMI-treated mice to lyse NK-sensitive YAC-1 targets in a 4-hr. ⁵¹Cr release assay (data not shown). FIG. 20 shows that in Ad/PSA-immunized mice, depletion of CD8+ T cells abrogated the protective effects of Ad/PSA. One hundred percent of mice in the CD8-depleted group developed tumors. However, tumor size in CD8-depleted mice was significantly smaller than those in control mice, suggesting that other effector components contribute to the antitumor effects of the PSA vaccine. Consistent with this observation, depletion of CD4+ T cells results in an increase in tumor outgrowth, indicating that this T cell population also contributes to the inhibition of RM11/PSA growth in Ad/PSA-immunized mice. NK cells do not appear to participate in the anti-tumor activity.

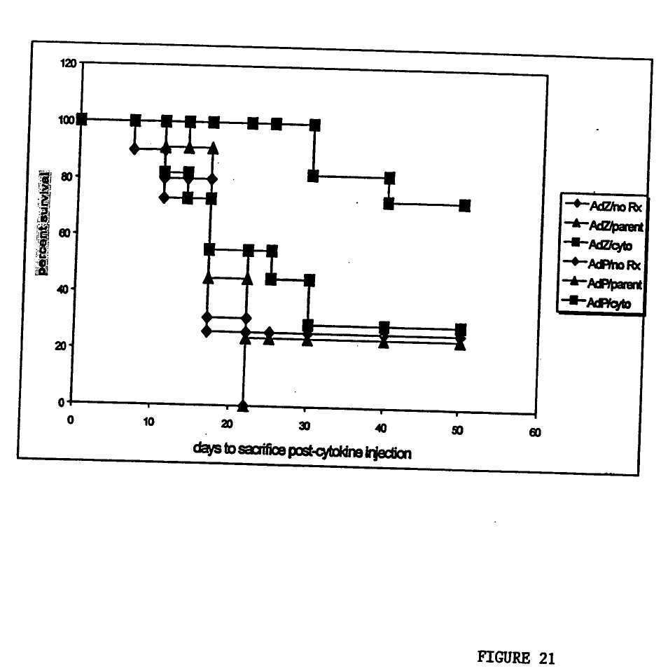

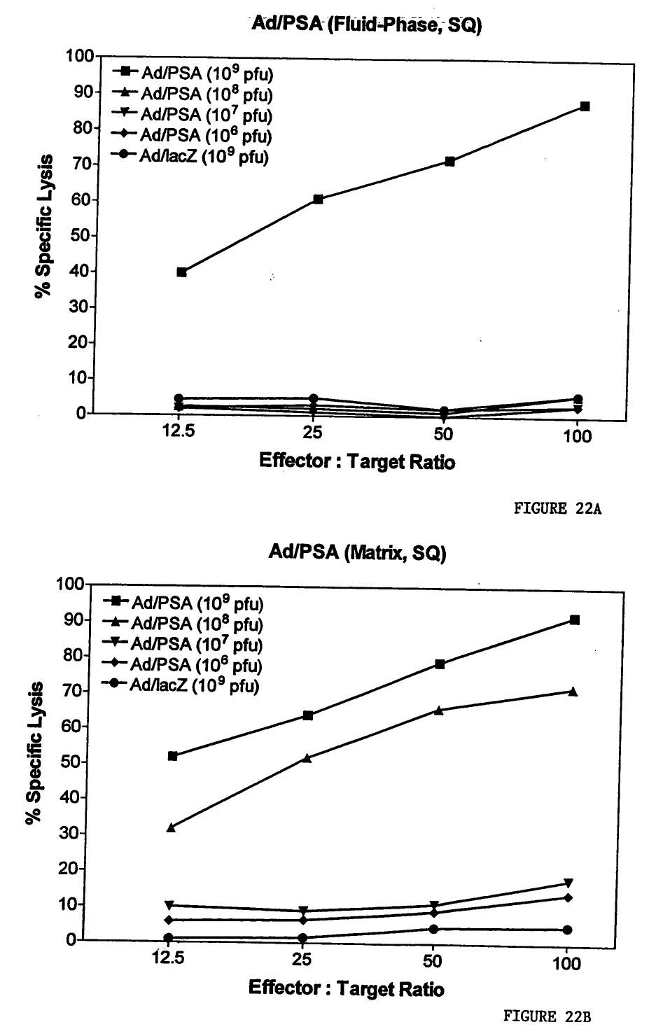

Ad/PSA treatment of established tumors Studies were performed to determine whether Ad/PSA immunization could effectively eliminate established RM11/PSA tumors. Mice were immunized with Ad/PSA 24 hours or 3 days after a sc. injection of 1×10⁵ clone E6 cells, and tumor growth and survival were monitored. At both timepoints, the results showed poor inhibition of tumor growth and protection in less than 20% of the animals. Since we had previously observed significant inhibition of RM11 growth after injecting an equal mix of recombinant ALVAC virus carrying genes for TNF-α, IL-12, and IL-2 in a Gelfoam matrix (in press, J. Natl. Cancer Institute), we tested the effects of Ad/PSA immunization in combination with intralesional injection of these recombinant viruses. Mice were immunized with Ad/PSA 3 days after the injection of E6 tumor cells. Seven days after Ad/PSA immunization, the cytokine-recombinant ALVAC vectors were injected into the E6 tumors, all of which had volumes from 500-1000 mm³. Combination therapy with Ad/PSA and intralesional ALVAC IL-2, IL-12, and TNF-α resulted in complete tumor regression in 8 of 10 mice (FIG. 21 A & B). In contrast, only 2 of 10 mice in each group receiving either Ad/PSA alone, Ad/PSA plus ALVAC parental virus, or Ad/lacZ plus ALVAC-cytokine had complete tumor regression.

In this experiment we show that a recombinant, replication-deficient adenovirus type 5 carrying the prostate-associated antigen PSA (Ad/PSA) induces a strong anti-PSA T cell response in a murine prostate cancer model. Ad/PSA-induced immunity is both long lasting and protective against prostate tumors expressing PSA. In addition, using intralesional cytokine therapy in combination with Ad/PSA immunization, we demonstrated the ability to destroy established prostate tumors that are otherwise resistant to treatment. Although PSA is currently a target antigen in clinical trials, this is the first published report showing that a viral vaccine encoding PSA can induce a protective and therapeutic PSA-specific anti-tumor response in a prostate cancer model.

Several investigators have reported preclinical studies evaluating the potential of immunotherapy in prostate cancer models. These studies have primarily employed the use of cytokine-secreting tumor cell vaccines, although the potential for transducing tumor cells with B7.1 or blocking CTLA-4 has been reported. (Kawakita M, et al., “Effect of canarypox (ALVAC)-mediated cytokine expression on murine prostate tumor growth” J Natl Cancer Inst 1997; 89:428-436;. Sanda M G, et al, “Demonstration of a rational strategy for human prostate cancer gene therapy”, J Urol 1994; 151:622-628; Vieweg J, et al., “Immunotherapy of prostate cancer in the Dunning rat model: use of cytokine gene modified tumor vaccines”, Cancer Res 1994; 54:1760-1765; Yoshimura I, “Cytokine mediated immuno-gene therapy in a rat prostate cancer model”, J Urol 1996; 155:510A; Kwon E D, et al., “Manipulation of T cell costimulatory and inhibitory signals for immunotherapy of prostate cancer”, Proc Natl Acad Sci USA 1997; 94:8099-9003) The reports have shown vaccine-dependent inhibition of primary tumor growth; however, the ability of the immunotherapy regimens to induce protective immunity was either not reported or protection was generally absent. Only one report showed protection against a secondary tumor challenge after immunization with IL-2-secreting tumor cells. Although protection against a secondary challenge was observed, neither the specificity of the response nor its mechanism of protection were characterized. (Vieweg J, et al., “Immunotherapy of prostate cancer in the Dunning rat model: use of cytokine gene modified tumor vaccines”, Cancer Res 1994; 54:1760-1765.) These data are contrasted by two other reports using the R3327 Dunning tumor model (Sanda M G, et al., “Demonstration of a rational strategy for human prostate cancer gene therapy”, J Urol 1994; 151:622-628; Yoshimura I, “Cytokine mediated immuno-gene therapy in a rat prostate cancer model” J Urol 1996; 155:510A), and one report using a mouse model which failed to observe protective immunity. (Kawakita M, et al., “Effect of canarypox (ALVAC)-mediated cytokine expression on murine prostate tumor growth”, J Natl Cancer Inst 1997; 89:428-436) The reason(s) for the differences among these studies is not known. We hypothesized that factors produced by the prostate cell lines, such as TGF-β, (Barrack E R, “TGF beta in prostate cancer: a growth inhibitor that can enhance tumorigenicity”, Prostate 1997; 31:61-70.) may have contributed to the absence of protective immunity in some studies. Thus, our strategy was to circumvent any possible immunomodulatory effects of tumor cell vaccines through the use of a recombinant viral vaccine carrying the gene for PSA.

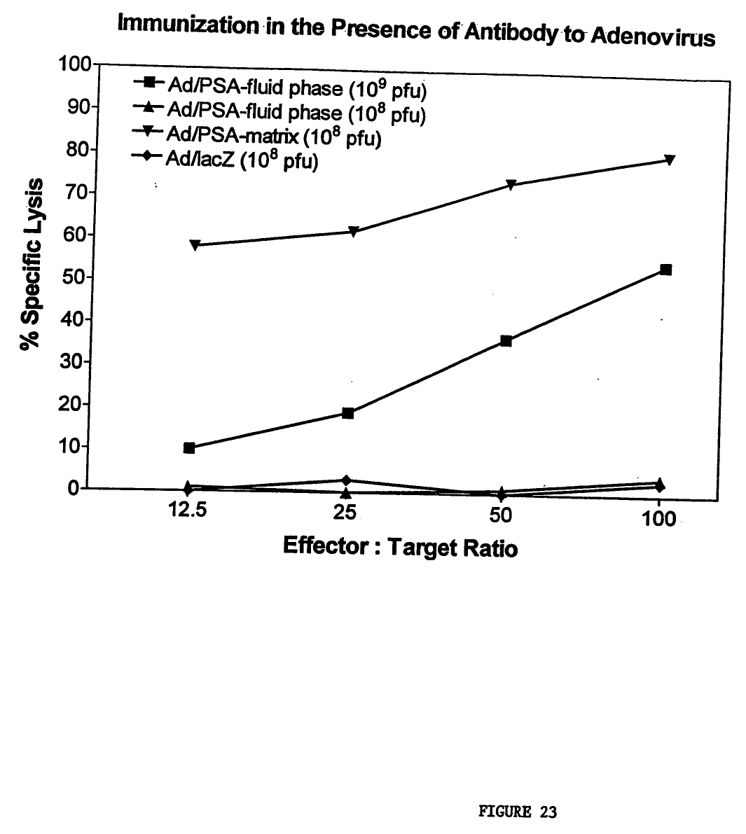

Our data clearly show the activation of PSA-specific protective immunity after Ad/PSA immunization. In vivo T cell depletion studies showed protection to be mediated largely by CD8+ effector T cells (FIG. 23), although modest, but reproducibly significant protection was observed in these CD8-depleted mice. Flow cytometry studies on the CD8-depleted mice showed that effective CD8+ T cell depletion was achieved, suggesting that other factors contribute to the protective effects of Ad/PSA immunization. In this regard, our data demonstrate that Ad/PSA also activates PSA-specific lymphoproliferation, implicating a CD4+ T cell response. Data shown in FIG. 23 demonstrate that CD4+ T cells contribute to vaccine-induced protective immunity. Depletion of these T cells after priming results in reproducible and significant reduction of protective immunity. It has been shown previously that CD4+ T cells can contribute to tumor immunity. In a protective response to tumors lacking MHC I expression, CD4+ T cells were the primary mediators of tumor rejection. (Levitsky H I, “In vivo priming of two distinct antitumor effector populations: the role of MHC class I expression” J Exp Med 1994; 179:1215-1224.) The mechanism by which CD4+ T cells mediate protection is not well defined. Previously reported mechanisms include CD40-mediated activation of antigen-presenting cells (Grewall I S, “The CD40 ligand. At the center of the immune universe?” Immunol Res 1997; 16: 59-70.), secretion of IL-2 to facilitate CTL expansion (Larsson E L, “Activation and growth requirements for cytotoxic and noncytotoxic T lymphocytes”, Cell Immunol 1984; 89:223-231.), or through direct killing via fas/fas-ligand induced apoptosis in an antigen-specific or bystander mechanism. (Wang R, “CD95-dependent bystander lysis caused by CD4⁺ T helper 1 effectors”, J Immunol 1996; 157:2961-2968.) Further studies are required to define the CD4+ T cell contribution to Ad/PSA-induced protective immunity. In addition to Ad/PSA-induced T cell responses, we also observed the induction of PSA-specific antibody. The data reported in this manuscript do not directly address a role for antibody, therefore, a contribution to the anti-tumor response by humoral immunity cannot be excluded.

We also evaluated the potential contribution of NK cells to Ad/PSA-induced immunity. Some reports characterizing anti-tumor activity induced by cytokine-secreting tumor cell vaccines demonstrated an important role for NK cells. These studies showed NK cells to be necessary for the elimination of CD8+ T cell-mediated control of both metastatic foci and secondary tumor challenge. (Levitsky H I, “In vivo priming of two distinct antitumor effector populations: the role of MHC class I expression”, J Exp Med 1994; 179:1215-1224; Tamura Y, “Immunotherapy of tumors with autologous tumor-derived heat shock protein preparations” Science 1997; 278:117-120). In contrast to these studies, others have shown no contribution by NK cells to cytokine-secreting tumor vaccine-induced protective immunity. (Dranoff G, et al., “Vaccination with irradiated tumor cells engineered to secrete murine granulocyte-macrophage colony-stimulating factor stimulates potent, specific, and long-lasting anti-tumor immunity” Proc Natl Acad Sci USA 1993; 90:3539-3543.) Our studies characterizing effector mechanisms in Ad/PSA-induced protective immunity show no apparent contribution by NK cells to the observed anti-tumor activity. At least two possibilities exist for why NK cells are not necessary in the anti-tumor response in our model. RM11/PSA express MHC class I molecules and are resistant to NK-mediated lysis in vitro (data not shown). This could prevent NK anti-tumor activity in vivo. Second, NK cells could participate in tumor lysis at a low level, but are entirely overshadowed by high levels of T cell anti-tumor activity.

Previous pre-clinical investigations on the potential of PSA as a tumor rejection antigen focused on non-prostate murine tumor cell lines transfected with human PSA. One report described PSA-expressing murine colonic adenocarcinoma cells that grew progressively in syngeneic immunocompetent mice, although tumor growth in these mice was inhibited relative to control-transfected tumor cells. The authors reported anti-PSA antibodies in 3 of 4 animals, but did not directly assess T cell immunity. (Karr J F, “The presence of prostate-specific antigen-related genes in primates and the expression of recombinant human prostate-specific antigen in a transfected murine cell line”, Cancer Res 1995; 55:2455-2462) Frelinger and associates showed that non-prostate PSA-expressing tumor cell vaccines induced PSA-specific protective immunity against challenge with a separate, non-prostate PSA-expressing cell line. (Wei C—W, et al., “Expression of human prostate-specific antigen (PSA) in a mouse tumor cell line reduces tumorigenicity and elicits PSA-specific cytotoxic T lymphocytes”, Cancer Immunol Immunother 1996; 42:362-368.) These investigators were able to detect the presence of anti-PSA CTL in vitro, but did not characterize their role in vivo. Their report shows that PSA can function as a tumor rejection antigen in a non-prostate model. Our studies extend these observations to a prostate tumor model, and in addition, demonstrate that Ad/PSA immunization in combination with intralesional cytokine therapy induces regression of large established prostate tumors. Furthermore, our studies employing use of a viral vaccine for tumor immunotherapy is more conducive to clinical application than immunizing with transduced tumor cells.

Cytokine therapy of established tumors has been shown to be effective in reducing or eliminating tumor burden in many models. (Lattime E C, “In situ cytokine gene transfection using vaccinia virus vectors”, Seminars in Oncology 1996; 23:88-100; Kovesdi I, “Adenoviral vectors for gene transfer”, Curr Opin Biotechnology 1997; 8:583-9; Gilboa E, “Immunotherapy of cancer with genetically modified tumor vaccines”, Seminars in Oncology 1996; 23:101-7) However, except in rare instances in isolated tumor models, this treatment is not effective beyond 3 to 7 days after tumor injection, and the mass of the tumors is very small, frequently being non-palpable. Those tumors that have been controlled beyond 7 days have been slow, non-aggressive tumors that have still had minimal volume. (Shimizu K, “Systemic administration of interleukin 2 enhances the therapeutic efficacy of dendritic cell-based tumor vaccines”, Natl Acad Sci 1999; 96:2268-73) There is only one previous report of immune control of established prostate tumors, which was achieved at 3 days post-inoculation (Vieweg J, et al., “Immunotherapy of prostate cancer in the Dunning rat model: use of cytokine gene modified tumor vaccines”, Cancer Res 1994; 54:1760-1765). Our studies extend these results in that we have demonstrated in an aggressive prostate tumor model the elimination of established tumors of up to 1000 mm³ by immunizing with Ad/PSA followed in 7 days by an intra-tumoral injection of ALVAC IL-12, ALVAC IL-2, and ALVAC TNF-α. We have also determined that injecting the recombinant ALVAC viruses in the contralateral flank has no therapeutic effect on tumor growth (data not shown). Moreover, mice rejecting tumor exhibited PSA-specific CTL activity in vitro whereas control mice (bearing tumor) did not (data not shown). Determining the mechanism by which intralesional but not opposite flank injection of recombinant ALVAC viruses enhances CTL activity and tumor destruction is currently being pursued.

In conclusion, we have found that Ad/PSA effectively activates antigen-specific protective immunity. CD8⁺ T cells, and to a lesser degree, CD4⁺ T cells, are responsible for protection against RM11/PSA tumor challenge, while NK cells do not appear to play a significant role. However, this strong protective response only poorly controlled established RM11/PSA. Using intralesional injection of recombinant ALVAC to deliver IL-12, IL-2, and TNF-α after immunization with Ad/PSA, we demonstrated elimination of large established tumors of unprecedented size. Our initial studies have been for proof-of-principle and are the first to demonstrate the ability of a viral vaccine to induce a cellular protective response against PSA. These observations have been the basis for FDA approval of clinical trials using Ad/PSA.

TABLE 1
PSA secretion into culture supernatant
fluid by transfected cell lines.

| Cell line | PSA secretion (ng/ml) |
|---|---|
| RM11/PSA clone E5 | 131.6 |
| RM11/PSA clone E6 | 0.5 |
| RM11/neo | 0.0 |
| P815/PSA | 15.5 |

TABLE 2
PSA production induced by infection with Ad/PSA. Supernatants
were analyzed by immunoassay. HEK 293 cells, due to their
genomic copy of the E1 gene, are permissive for replication
of the E1 deletion mutant Ad/PSA. Since RM11 cells do not
express E1, they are non-permissive.

| Tumor cell | State of infection | PSA secretion (ng/ml) |
|---|---|---|
| HEK 293 | permissive | 2711.2 |
| RM11 | non-permissive | 100.6 |

### Example 4

In the FIG. 23, entitled “Immunization in the Presence of Antibody to Adenovirus”, we utilized Gelfoam© to overcome antibody inhibition of the immunization process. The mice were pre-immunized with adenovirus carrying the lacZ gene (gene for μ-galactosidase). Two weeks later animals were immunized with either fluid phase adenovirus PSA or adenovirus PSA in Gelfoam©. As you will see from the data, immunization with Ad/PSA matrix at 10⁸ pfu provided excellent immunization whereas immunization with either 10⁸ or 10⁹ Ad/PSA in fluid phase resulted in significantly reduced immunization. A control immunization with Ad/lacZ was at background levels. These data demonstrate the potential of a collagen carrier to overcome the inhibitory effects of neutralizing antibody to viruses.

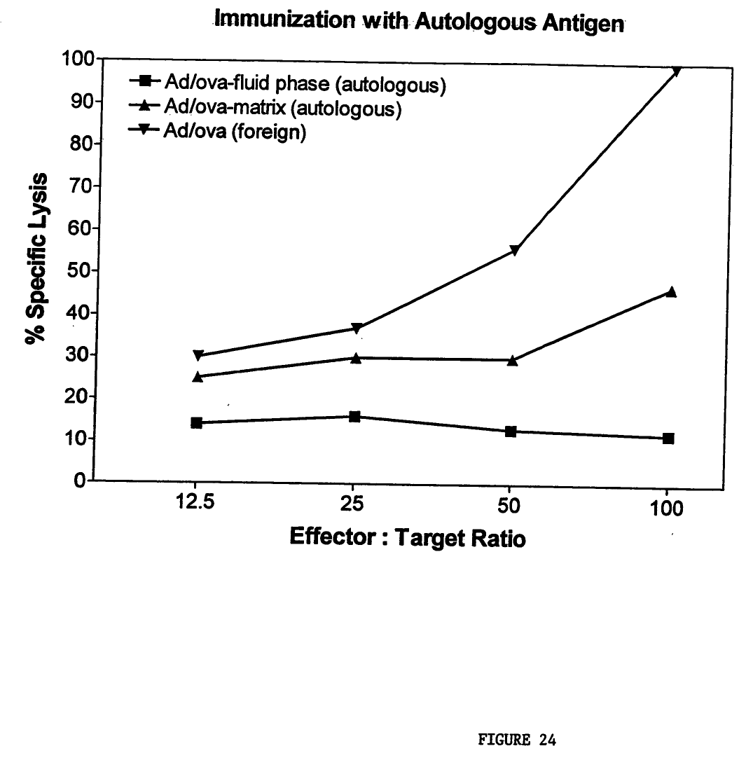

In the FIG. 24, “Immunization with Autologous Antigen”, we demonstrate that adenovirus in Gelfoam© is a much better immunogen than fluid phase adenovirus in a setting where the immunizing antigen is an autologous antigen. A transgenic mouse expressing ovalbumin (OVA) was used as a model for autologous antigen immunization. In this model ovalbumin is expressed under the control of the rat insulin promoter, which induces expression primarily in the pancreas and kidney. In this setting fluid phase adenovirus was unable to immunize the mice (induce cytotoxic T lymphocyte activity). Adenovirus administered in fluid phase in normal C57BL/6 mice (Ad/ova foreign antigen system) was a strong immunogen. In this later setting, the ovalbumin is a foreign antigen and induction of cytotoxic T lymphocyte activity is much easier. In contrast to fluid phase adenovirus in the autologous setting, Ad/ova administered in Gelfoam© induced significant cytotoxic T lymphocyte activity. These data further demonstrate the utility of the Gelfoam© in inducing cytotoxic T lymphocyte activity in a setting where tolerance is expected.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

### Example 5

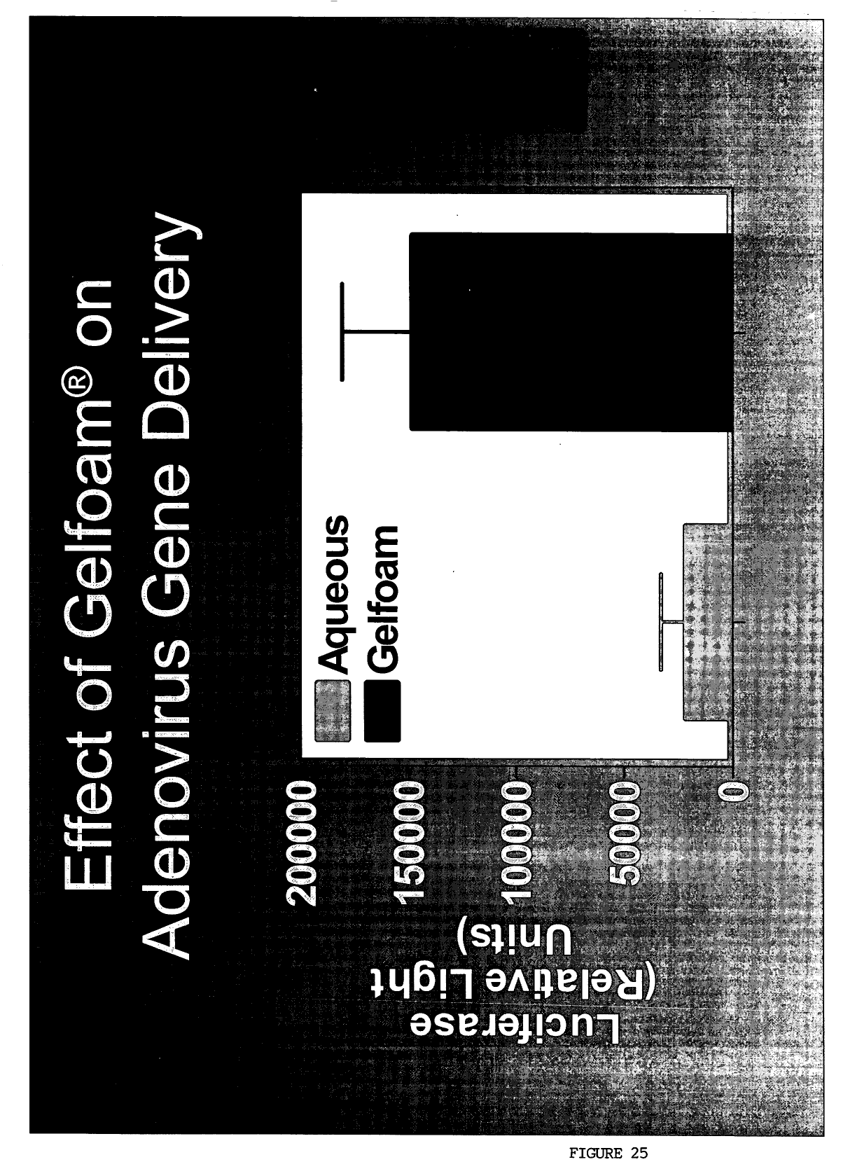

FIGS. 25-36 represent additional data obtained using the methods of the invention.

FIG. 25. Gelfoam augments adenovirus mediated gene delivery.

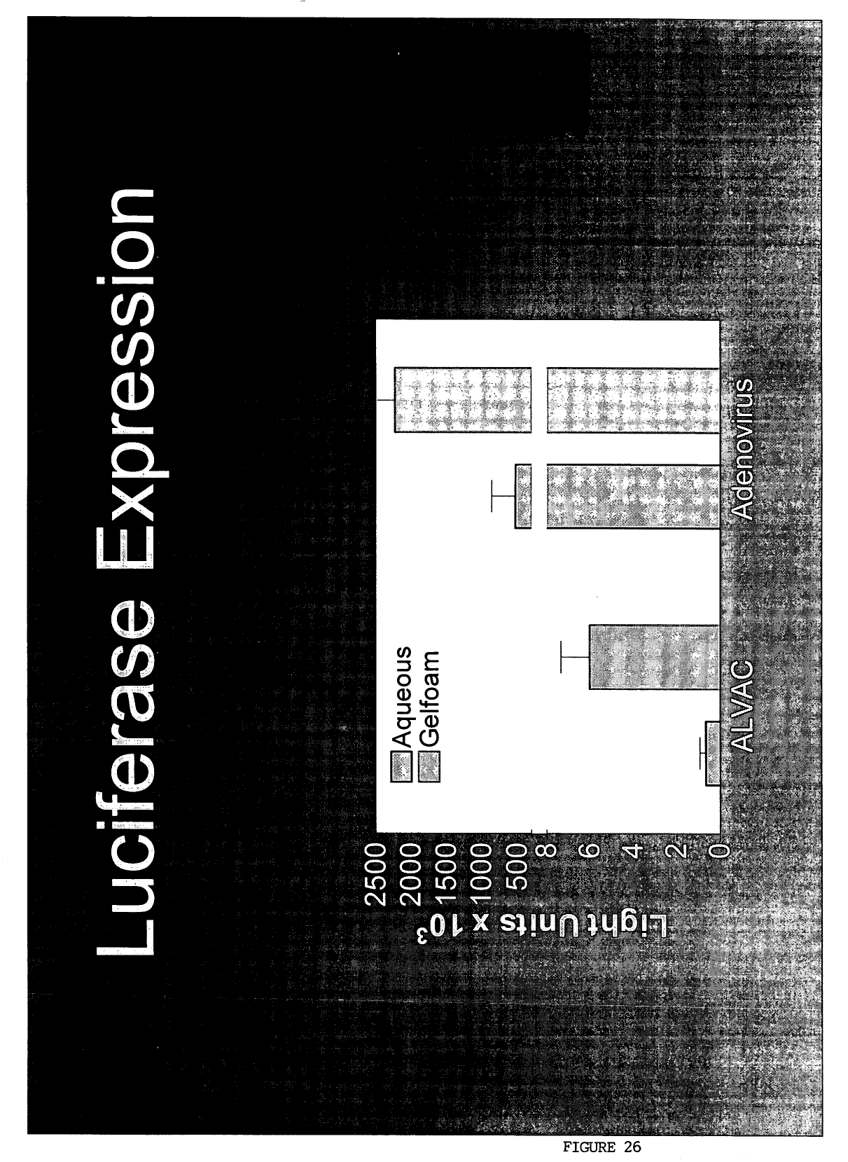

FIG. 26. Comparison of the effects of Gelfoam on gene delivery with either ALVAC (canarypox virus) and type 5 non-replicative Adenovirus

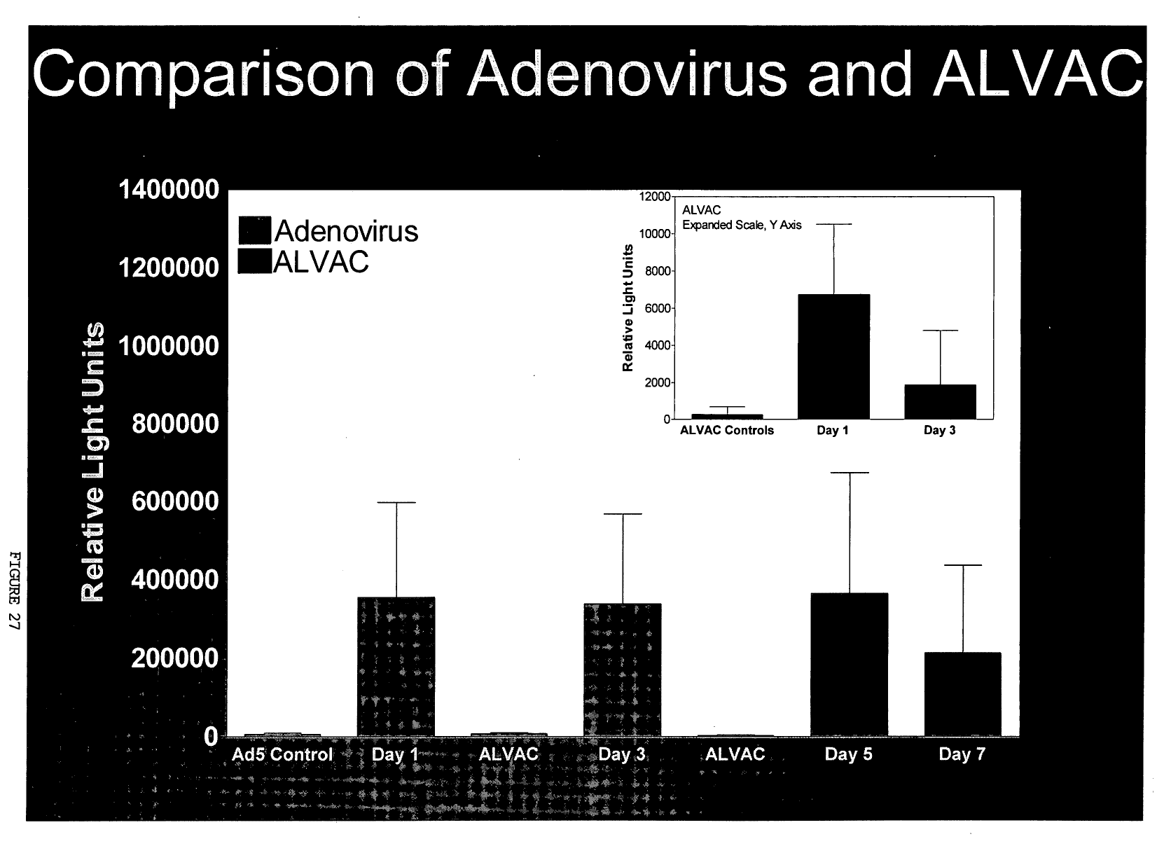

FIG. 27. Time course of expression with Gelfoam for both ALVAC and Adenovirus

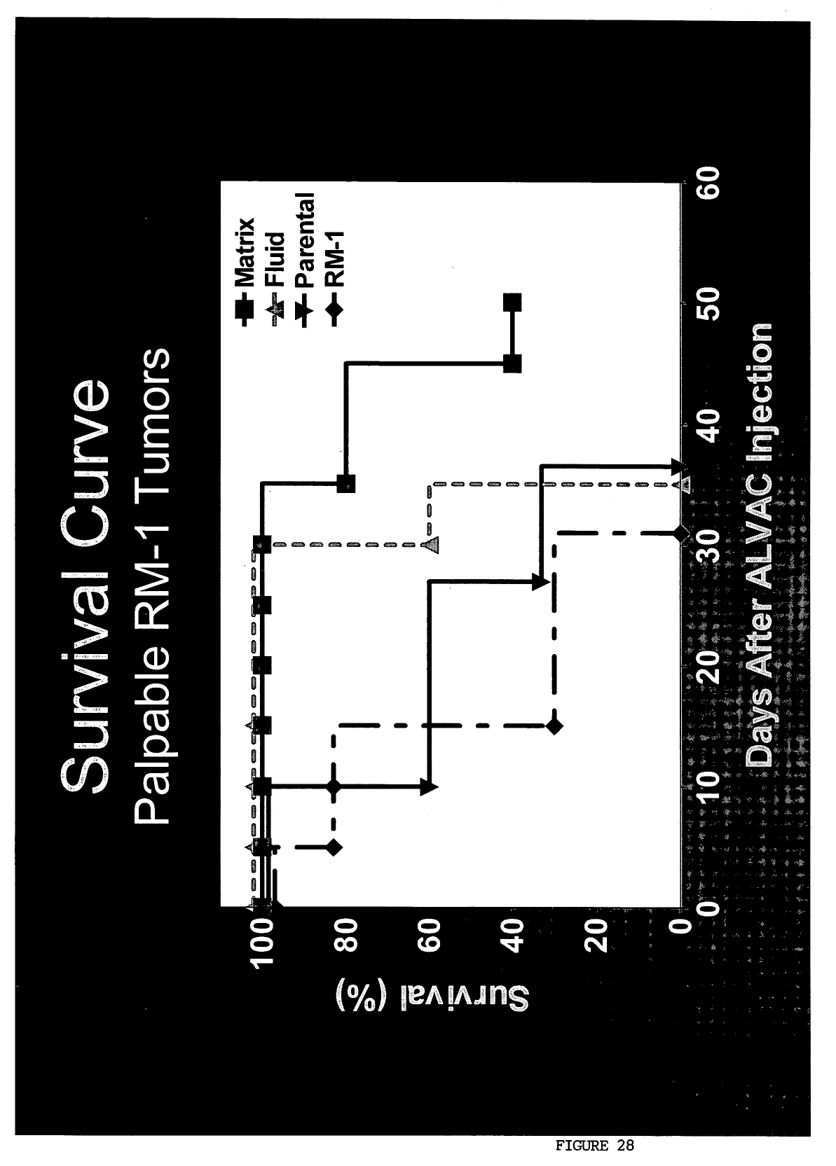

FIG. 28. Effect of Gelfoam on the antitumor activity of ALVAC carrying the genes for IL-2, IL12 and TNF. Tumor target is RM-1, a prostate cancer cell line. Only virus administered with Gelfoam resulted in effective antitumor activity (complete inhibition of tumor outgrowth in 40% of mice).

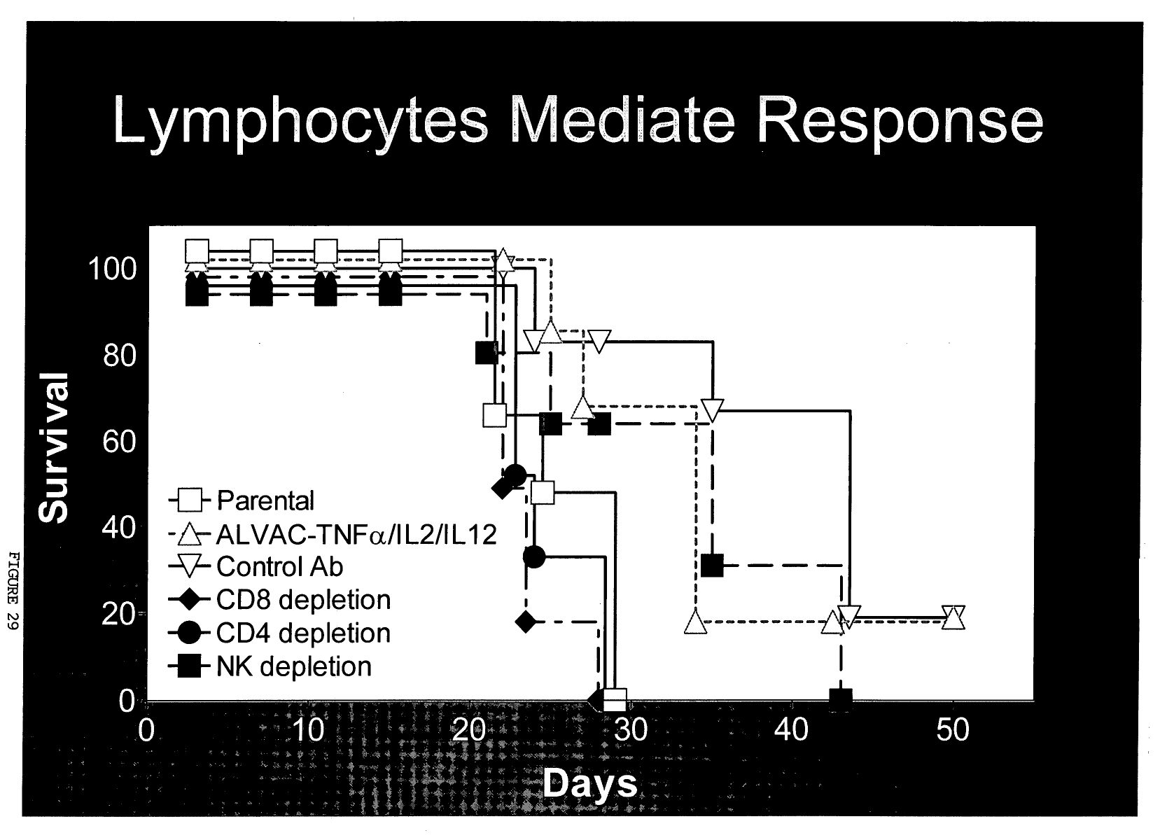

FIG. 29. Determination that CD4+ and CD8+ T cells mediate the antitumor effects induced by combination Gelfoam and ALVAC-IL2,IL12,TNF treatment.

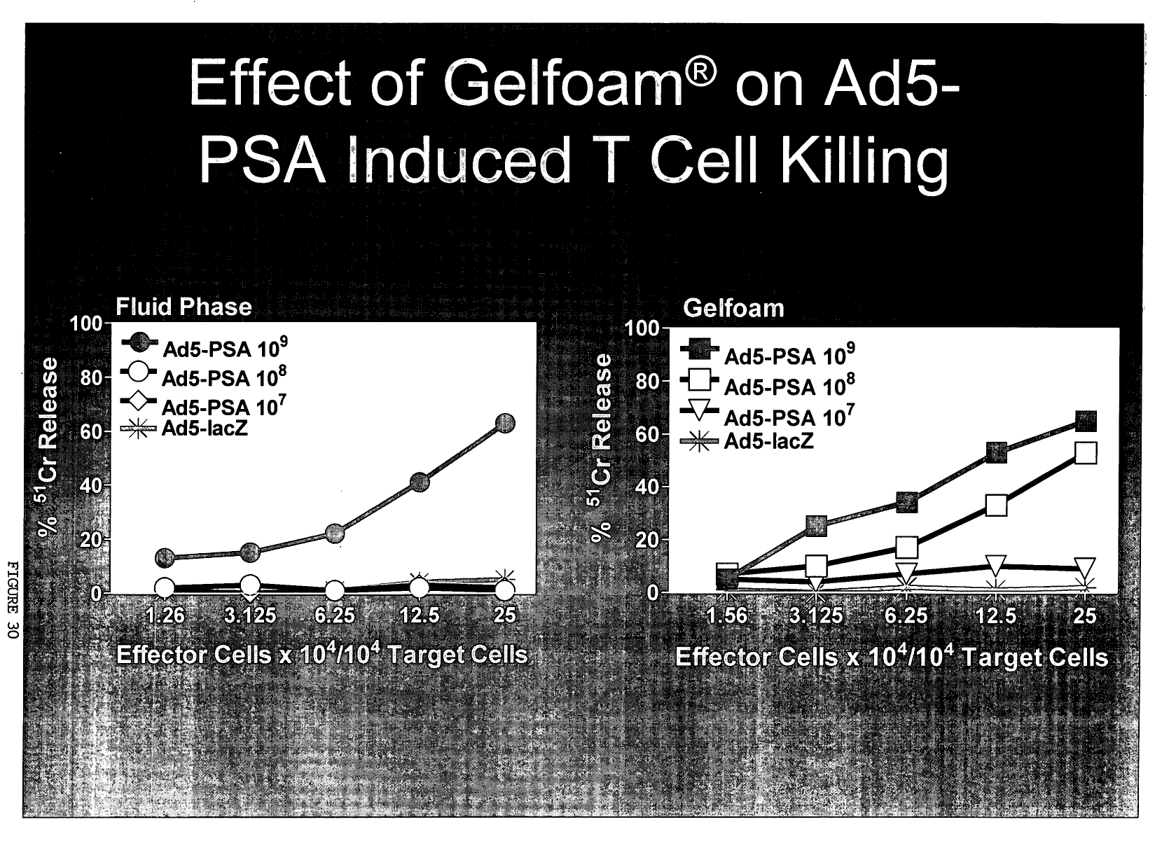

FIG. 30. Independent confirmation that Gelfoam augments T cell activation. Mice were immunized with recombinant Adenovirus carrying the gene for ovalbumin (Ad5-mOVA), a common antigen used to measure T cell activation. In this study varying doses of Ad5-mOVA were injected in aqueous phase or with Gelfoam. Effective lytic activity was observed at lower virus doses with Gelfoam.

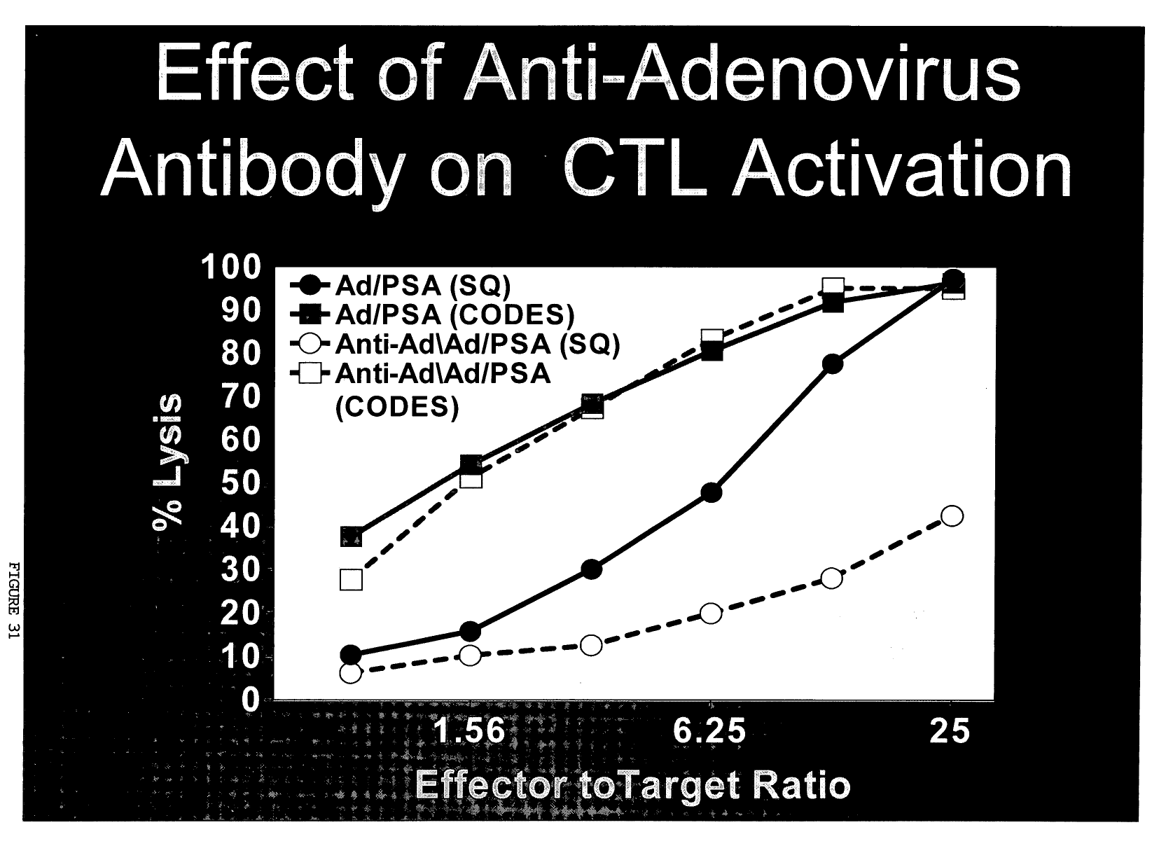

FIG. 31. Gelfoam has the ability to overcome the inhibitory effects of circulating antibody to Adenovirus on T cell activation. Mice were preimmunized with parental non-replicative adenovirus. Once antibody concentrations to adenovirus were confirmed, mice were immunized with Ad5-mOVA with or without Gelfoam. When Gelfoam was included, no inhibitory effects on T cell activation were observed.

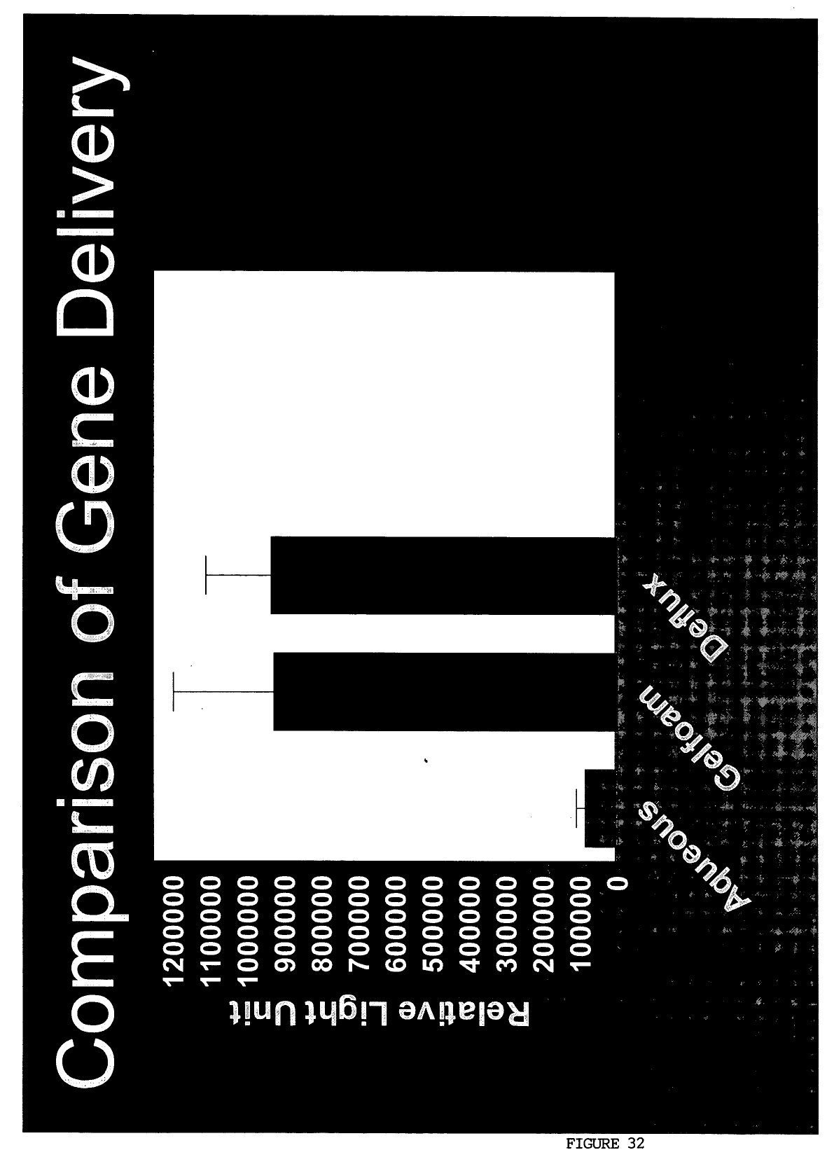

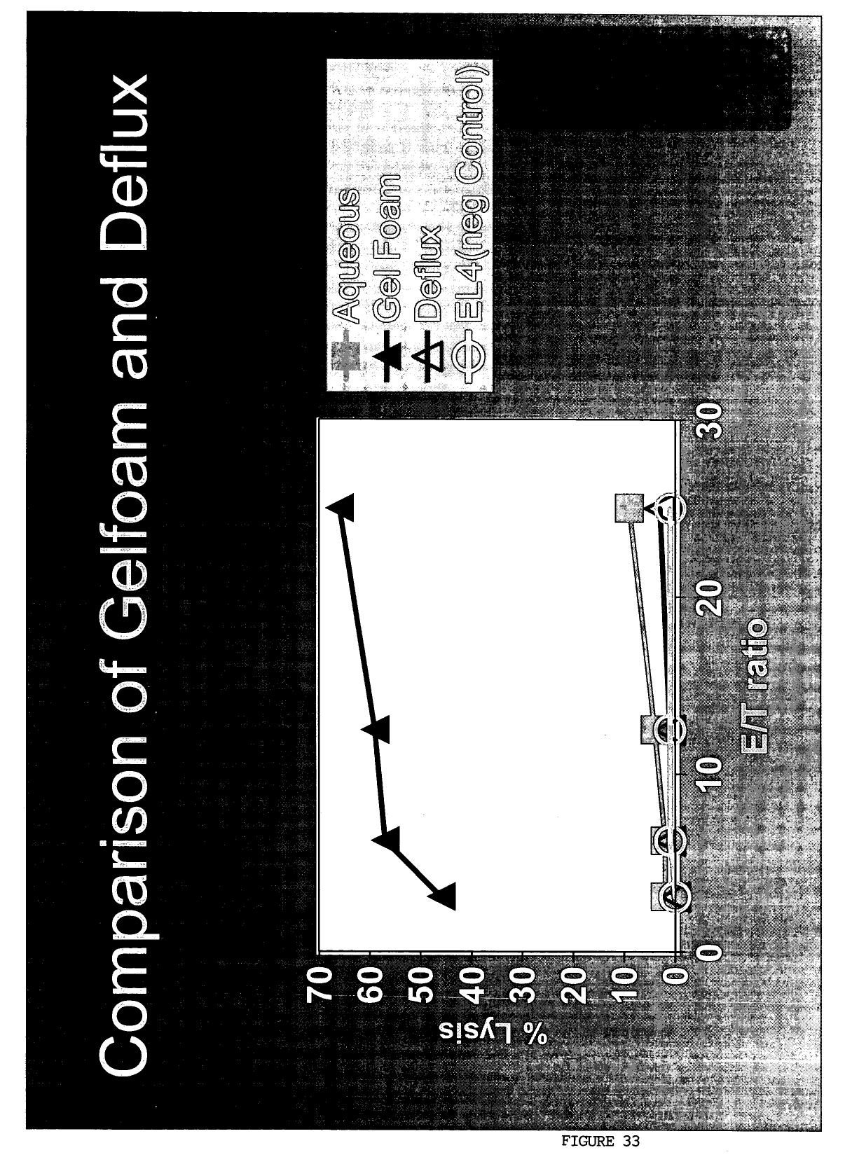

FIG. 32. Comparison of the gene delivery capacity of aquaeous phase, Gelfoam, and DeFlux to augment gene delivery. Deflux is a combination of cross-linked dextran and hyaluronidase. The non-collagen based matrix augments gene delivery at a level equivalent to that of Gelfoam

FIG. 33. While Deflux also augments gene delivery, it does not enhance T cell activation. Only Gelfoam augments both gene delivery and T cell activation.

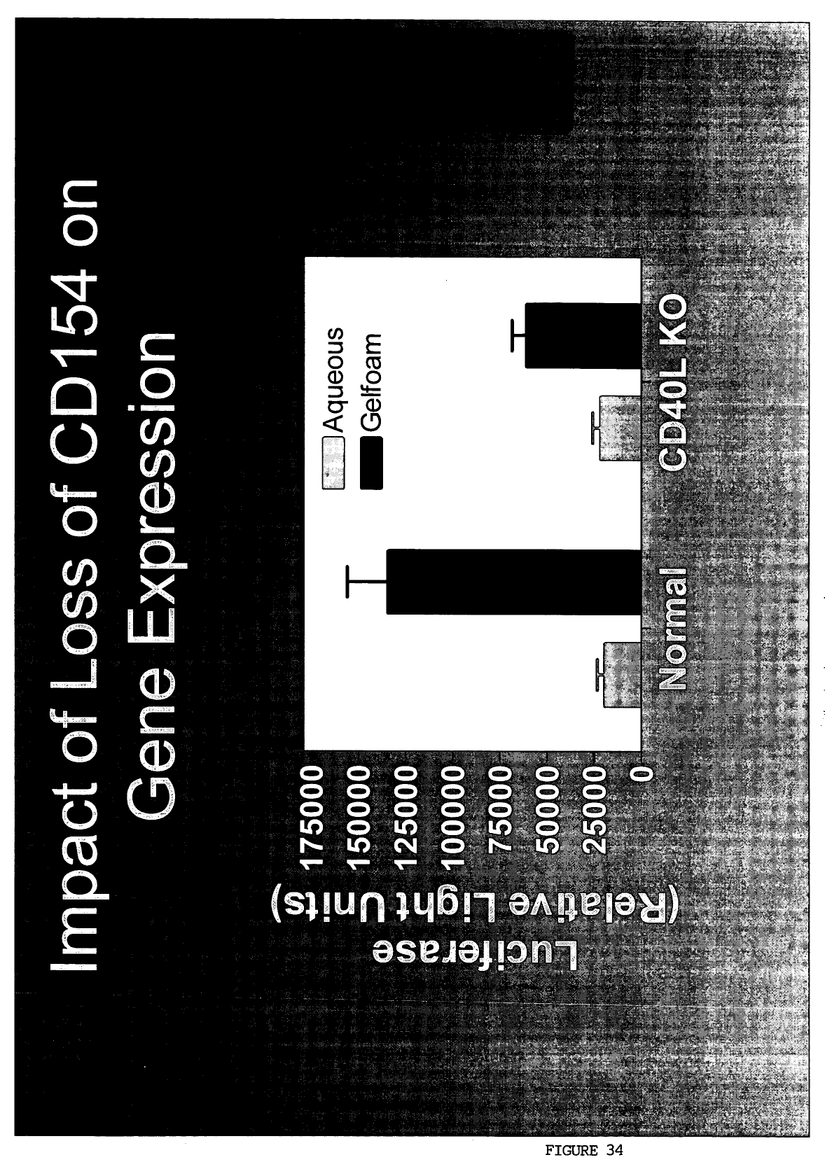

FIG. 34. Tested the ability of Gelfoam to augment gene delivery in CD154 knockout mice. CD 154 is an important TNF family member that is known to augment T cell responses and induce isotype switching in B cell responses. The data show that although gene expression is lower in CD 154 knockout mice, Gelfoam still augments gene delivery. The lower gene expression in CD 154 mice is consistent in many experiments, suggesting that this ligand is important in gene delivery.

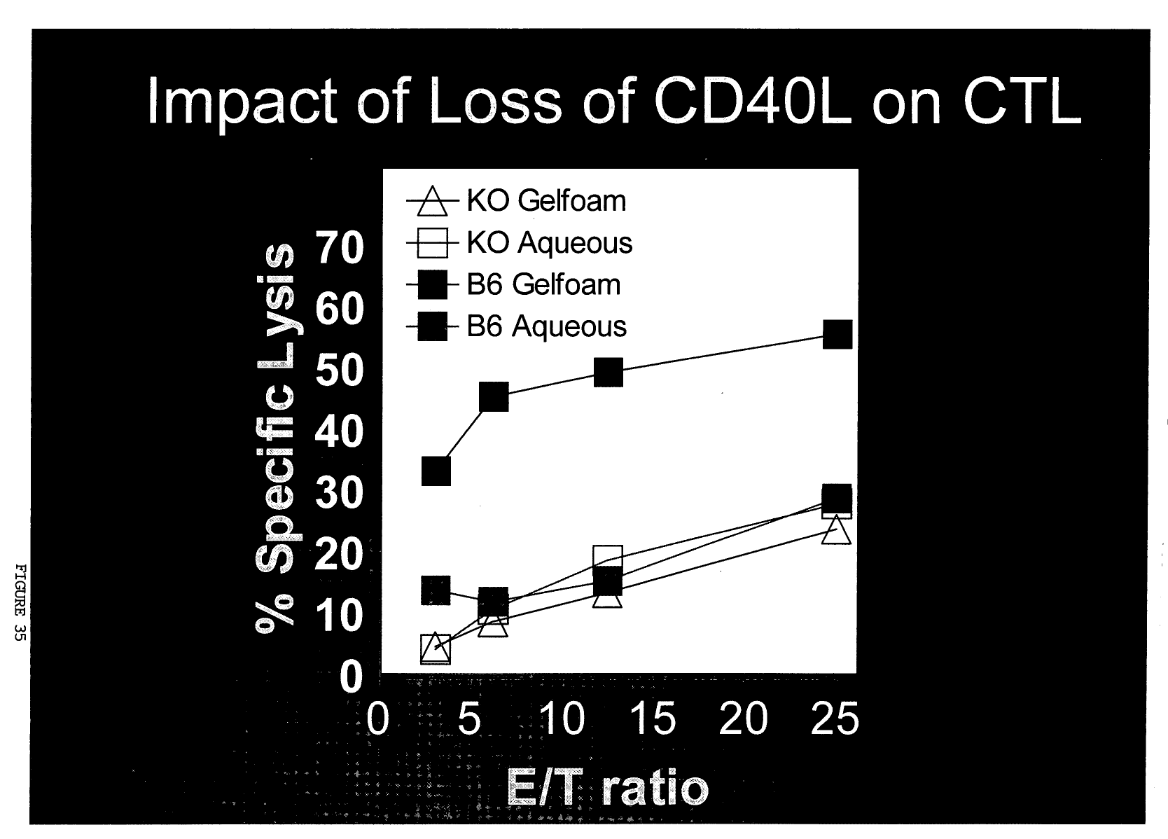

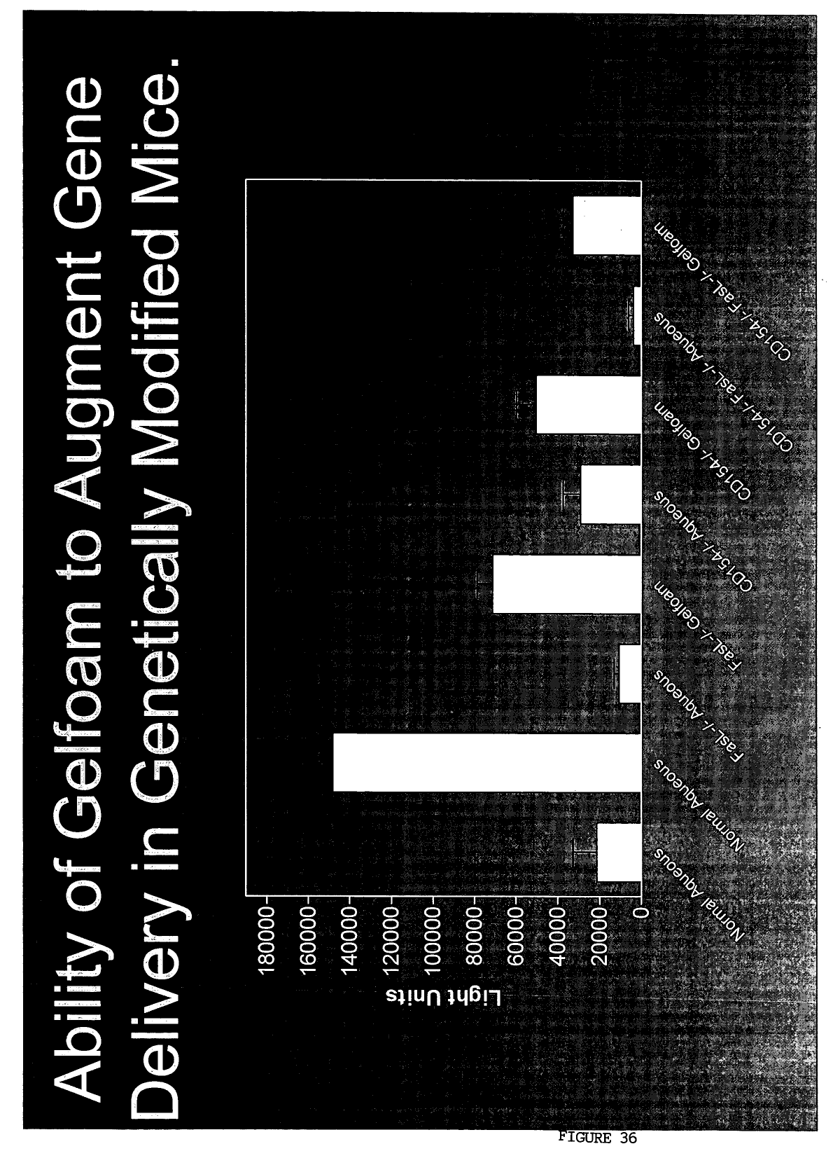

FIG. 35. Tested the ability of Gelfoam to augment T cell activation in CD154 knockout mice. Gelfoam lost its ability to enhance T cell activation in these mice (CD154 knockouts).

FIG. 36. Tested the ability of Gelfoam to augment gene delivery in genetically modified mice, including CD 154−/−, FasL−/−, and double knockout mice (CD 154−/− & FasL−/−). Note some loss of gene expression in single knockout mice but complete loss in double knockout mice.

What is claimed is:

1. A method for stimulating an antigen specific immune response in an animal comprising:
introducing to a cell in said animal a nucleotide expression system, said expression system comprising:
a nucleotide sequence which encodes an antigenic amino acid sequence, a promoter, and a transcription termination signal;
said nucleotide system associated with a collagen carrier.

2. The method of claim 1 wherein said nucleotide expression system comprises a viral vector.

3. The viral vector of claim 2 wherein said viral vector is a virus selected from the group consisting of retrovirus, adeno-associated virus, herpes virus, lenta virus, canary pox virus, and Epstein Barr virus.

4. The vector of claim 2 further comprising an expression system which encodes a marker selection gene for selection of transformants.

5. The vector of claim 4 wherein said marker selection gene comprises a gene selected from the group consisting of:
beta galactosidase, green fluorescent protein, and/or luciferase.

6. The method of claim 1 wherein said antigenic amino acid sequence comprises prostate specific antigen.

7. The method of claim 1 wherein said expression system carrier mixture is introduced by injection.

8. The method of claim 7 wherein said method of introduction includes intratissue injection.

9. The method of claim 1 wherein said cells are protected against later challenge.

10. The method of claim 9 wherein said method is used to protect against challenge by cancer cells.

11. The method of claim 10 wherein said cancer cells are those associated with prostate cancer.

12. The method of claim 1 wherein said collagen carrier comprises fibrous collagen.

13. The method of claim 1 wherein said collagen is extracted from tendon or dermal collagen.

14. The method of claim 1 wherein said collagen carrier comprises mineralized collagen.

15. The method of claim 1 wherein said collagen is selected from the group consisting of Type I through Type IXX.

16. The method of claim 1 wherein said collagen carrier comprises purified skin gelatin.

17. The method of claim 16 wherein said collagen comprises Gelfoam®.

18. The method of claim 1 further comprising the step of:
introducing to said cell, after challenge, a second nucleotide expression system, said second nucleotide expression system comprising nucleotide sequence encoding a cytokine, a promoter, and a transcription termination signal; said nucleotide system associated with a collagen carrier.

19. The method of claim 18 wherein said cytokine is selected from the group consisting of: IL-12, IL-2, and TNF-α.

20. A method for inducing an antigen specific immune response in an animal comprising:
introducing to a cell in said animal an antigen composition, said composition comprising: an antigenic amino acid, and a collagen carrier, wherein said carrier and said protein are associated.

21. The method of claim 20 wherein said antigen is prostate specific antigen.

22. The method of claim 20 wherein said collagen carrier comprises fibrous collagen.

23. The method of claim 20 wherein said collagen is extracted from tendon or dermal collagen.

24. The method of claim 20 wherein said collagen carrier comprises mineralized collagen.

25. The method of claim 20 wherein said collagen is selected from the group consisting of Type I through Type IXX.

26. The method of claim 20 wherein said collagen carrier comprises purified skin gelatin.

27. The method of claim 26 wherein said collagen comprises Gelfoam®.

28. The method of claim 20 further comprising the step of:
introducing to said cell after challenge with a tumor, a second nucleotide expression system, said second nucleotide expression system comprising nucleotide sequence encoding a cytokine, a promoter, and a transcription termination signal;
said nucleotide system associated with a collagen carrier.

29. The method of claim 28 wherein said cytokine is selected from the group consisting of: IL-12, IL-2, and TNF-α.

30. An immunogenic composition for stimulating a specific antigen immune response comprising:
a nucleotide expression system comprising a nucleotide sequence which encodes an antigenic protein, a promoter, and a transcription termination signal;
and a collagen carrier, wherein said carrier and expression system are associated.

31. The vaccine of claim 30 wherein said antigen is a tumor specific antigen.

32. The vaccine of claim 30 wherein said antigen is prostate specific antigen.

33. An immunogenic composition for stimulating a specific antigen immune response comprising:
an antigenic protein and
a collagen carrier, wherein said carrier and expression system are associated.

34. A cancer vaccine which provides a protective and synergistic response to challenge with introduction of cytokines, comprising:
a nucleotide expression system comprising a nucleotide sequence which encodes a timor specific antigen, a promoter, and a transcription termination signal;
and a collagen carrier, wherein said carrier and expression system are associated.

35. The vaccine of claim 34 wherein said antigen is prostate specific antigen.

36. A method of protecting against and or eliminating cancer cells comprising:
introducing to said cells a vaccine composition comprising a cancer specific antigen and a collagen carrier wherein said antigen is associated with said carrier and thereafter, upon challenge,
administering a cytokine composition.

37. The method of claim 36 wherein said antigen is a nucleotide expression system, said expression system comprising a nucleotide sequence which encodes a cancer specific antigen, a promoter, and a transcription termination signal.

38. The method of claim 36 wherein said cytokine composition comprises:
a nucleotide expression system, said expression system comprising: a nucleotide sequence which encodes a cytokine, a promoter and a transcription termination signal; and
a collagen carrier.

39. The method of claim 36 wherein said cytokine is selected from the group consisting of: tissue necrosis factor and interleukin.

40. The method of claim 36 wherein said antigen is prostate specific antigen.
